SEQUENCE LISTING (28 sequences)

SEQ ID NO: 1 - protein, 17 aa, Artificial Sequence
  Synthetic
MSSSSWLLLSLVAVTAA

SEQ ID NO: 2 - protein, 598 aa, Artificial Sequence
  Synthetic
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 3 - protein, 262 aa, Artificial Sequence
  Synthetic
GGGGSGGGGSGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 232 aa, Artificial Sequence
  Synthetic
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 20 aa, Artificial Sequence
  Synthetic
MKWVTFISLLFLFSSAYSGS

SEQ ID NO: 6 - protein, 220 aa, Artificial Sequence
  Synthetic
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 631 aa, Artificial Sequence
  Synthetic
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGSGGGGSGGGGSGGGGSGGGGSGGGGSGGGGS

SEQ ID NO: 8 - protein, 448 aa, Artificial Sequence
  Synthetic
QMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 447 aa, Artificial Sequence
  Synthetic
QMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 10 - protein, 633 aa, Artificial Sequence
  Synthetic
GGSGGGGSGGGGSGGGGSGGGGSGGGGSGGGGSEFQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 11 - protein, 597 aa, Homo sapiens
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 12 - protein, 219 aa, Artificial Sequence
  Synthetic
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 219 aa, Artificial Sequence
  Synthetic
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 12 aa, Artificial Sequence
  Synthetic
EPKSCDKTHTCP

SEQ ID NO: 15 - protein, 598 aa, Artificial Sequence
  Synthetic
QSTIEEQAKTFLDFFDSQAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 16 - protein, 859 aa, Artificial Sequence
  Synthetic
QSTIEEQAKTFLDFFDSQAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGGSGGGGSGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 598 aa, Artificial Sequence
  Synthetic
QSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 18 - protein, 859 aa, Artificial Sequence
  Synthetic
QSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGGSGGGGSGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 597 aa, Artificial Sequence
  Synthetic
QSIIEEQAKTFLDKFNAEAEDLFYQSSLASWNYNTNITEENVQNMNNAGDKWSAFLKEQSTLAQMYPLQEIQNLQVKLQLQALHQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 20 - protein, 858 aa, Artificial Sequence
  Synthetic
QSIIEEQAKTFLDKFNAEAEDLFYQSSLASWNYNTNITEENVQNMNNAGDKWSAFLKEQSTLAQMYPLQEIQNLQVKLQLQALHQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAGGGGSGGGGSGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 597 aa, Artificial Sequence
  Synthetic
QSTIEEQVKTFLDNFNHKAEDLFYQSSLASWNYNTNITEENVQNMNNAGDKWSAFLKEQSTFAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 22 - protein, 858 aa, Artificial Sequence
  Synthetic
QSTIEEQVKTFLDNFNHKAEDLFYQSSLASWNYNTNITEENVQNMNNAGDKWSAFLKEQSTFAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAGGGGSGGGGSGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 598 aa, Artificial Sequence
  Synthetic
QSTIEEQAKWFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 24 - protein, 859 aa, Artificial Sequence
  Synthetic
QSTIEEQAKWFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGGSGGGGSGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 56 aa, Artificial Sequence
  Synthetic
DKEWILQKIYEIMRLLDELGHAEASMRVSDLIYEFMKKGDERLLEEAERLLEEVER

SEQ ID NO: 26 - protein, 317 aa, Artificial Sequence
  Synthetic
DKEWILQKIYEIMRLLDELGHAEASMRVSDLIYEFMKKGDERLLEEAERLLEEVERGGGGSGGGGSGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 64 aa, Artificial Sequence
  Synthetic
NDDELHMLMTDLVYEALHFAKDEEIKKRVFQLFELADKAYKNNDRQKLEKVVEELKELLERLLS

SEQ ID NO: 28 - protein, 325 aa, Artificial Sequence
  Synthetic
NDDELHMLMTDLVYEALHFAKDEEIKKRVFQLFELADKAYKNNDRQKLEKVVEELKELLERLLSGGGGSGGGGSGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2023/0129210 A1
(43) Pub. Date: Apr. 27, 2023

(54) BINDING PROTEINS USEFUL AGAINST ACE2-TARGETED VIRUSES

(71) Applicant: The University of North Carolina at Chapel Hill, Chapel Hill, NC (US)

(72) Inventors: Samuel Lai, Chapel Hill, NC (US); Karthik Tiruthani, Chapel Hill, NC (US); Carlos Alberto Cruz Teran, Chapel Hill, NC (US)

(21) Appl. No.: 17/914,000

(22) PCT Filed: Apr. 5, 2021
(86) PCT No.: PCT/US2021/025787

§ 371 Date: Sep. 23, 2022

Related U.S. Application Data
(60) Provisional application No. 63/004,823, filed on Apr. 3, 2020.

(51) Int. Cl. C07K 14/47; A61K 47/68

(52) U.S. Cl. CPC C07K 14/47; A61K 47/68; C07K 2317/76; C07K 2319/31; C07K 2319/32; C07K 2319/40; C07K 2319/30

(57) ABSTRACT

Described herein are binding proteins useful against ACE2-targeted viruses (e.g., SARS-CoV and SARS-CoV-2, etc.), and methods of using them. These binding proteins may include an extracellular portion of angiotensin-converting enzyme 2 (ACE2), excluding the collectrin domain, and a flexible polypeptide flexible linker coupling the ACE2 portion to a fragment crystallization (Fc) domain. These binding proteins dimerize, and the flexible linker may be chosen to be sufficiently long to permit concurrent interaction with multiple Spike (S) proteins on the ACE2-targeted virus.

### CROSS REFERENCE TO RELATED APPLICATIONS

This patent application claims priority to U.S. provisional patent application No. 63/004,823, titled “BINDING PROTEINS USEFUL AGAINST SARS-LIKE CORONAVIRUSES,” and filed on Apr. 3, 2020, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are binding proteins that bind to severe acute respiratory syndrome coronaviruses (SARS-CoV and SARS-CoV-2). These binding proteins can be flexibly-linked. ACE2 decoys, which can be used in pharmaceutical compositions to treat a subject suffering from SARS-CoV and/or SARS-CoV-2 infections, as well as methods of using them.

### BACKGROUND

The SARS-CoV-2 pandemic has had an unprecedented disruptive global societal and economic impact and has marked the third known zoonotic introduction of a highly pathogenic coronavirus into the human population. Although the previous coronavirus SARS-CoV and MERS-CoV epidemics raised awareness of the need for clinically available therapeutic or preventive interventions, to date, no treatments with proven efficacy are available. The development of effective intervention strategies relies on the knowledge of molecular and cellular mechanisms of coronavirus infections, which highlights the significance of studying virus-host interactions at the molecular level to identify targets for antiviral intervention and to elucidate critical viral and host determinants that are decisive for the development of severe disease.

The mucosa barrier plays an important potential protective role as a barrier to prevent foreign matter from entering the body. The mucosal barrier may be further enhanced by local immunity that allows a robust immune system response to occur at mucosal membranes of the intestines, the urogenital tract and the respiratory system, i.e., surfaces that are in contact with the external environment. The mucosal immune system may provide protection against pathogens but maintains a tolerance towards non-harmful commensal microbes and benign environmental substances. Since the mucosal membranes are the primary contact point between a host and its environment, a large amount of secondary lymphoid tissue is found here. The mucosa-associated lymphoid tissue, or MALT, provides a critical element of the mucosal immune response. The mucosal immune system provides three main functions: serving as the body's first line defense from antigens and infection, preventing systemic immune responses to commensal bacteria and food antigens (primarily food proteins in the gut-associated lymphoid tissue, so-called oral tolerance), and regulating appropriate immune responses to pathogens encountered on a daily basis.

Unfortunately, the mucosal immune response may be inadequate, and it is often difficult to elicit the necessary immune response for sufficient duration. See, e.g., U. S. Patent Publication No. 2015/0284451. Although some antibodies have been shown to interact with mucins to adhesively crosslink individual antibody-coated pathogens to mucins and thereby immobilizing them in mucus (a process frequently referred to as muco-trapping), it would be beneficial to provide antibodies or antibody constructs having further improved ability to more effectively prevent foreign matter, including viruses, from permeating through mucus to reach target cells. In particular, it would be helpful to provide binding proteins such as antibodies that may assist in agglutination and/or enchainment of foreign entities together in a manner that limits their effective permeation through mucus.

### SUMMARY OF THE DISCLOSURE

Described herein are methods and compositions for enhancing agglutination, enchainment and/or muco-trapping of one or more ACE2-targeted viruses (e.g., SARS-CoV and SARS-CoV-2, etc.), reducing the fraction of ACE2-targeted viruses that can permeate through mucus. In particular, described herein are engineered binding proteins useful against ACE2-targeted viruses. These binding proteins may be polyvalent for ACE2-targeted viruses and may include two coronavirus-binding regions that are each flexibly linked by a flexible polypeptide linker to an Fc domain. The linkers may be sufficiently long and flexible so that both coronavirus-binding regions can bind to target (e.g., spike proteins), simultaneously.

For example, described herein are angiotensin-converting enzyme 2 (ACE2) Immunoglobulin (IgG) hybrid binding proteins (referred to herein as flexibly linked ACE2 decoys), that dimerize and have picomolar affinity for ACE2-targeted viruses, including in particular SARS-CoV-2. These proteins may be engineered for “muco-trapping”, and may be used to treat or prevent SARS-CoV (e.g., SARS-CoV-2) infection, for example, for topical immunotherapy against ACE2-targeted viruses including SARS-CoV-2. These molecules may generally be formed by coupling two or more extracellular portion(s) of ACE2 (e.g., a portion of soluble angiotensin-converting enzyme 2) to a Fc portion using a flexible linker, such as but not limited to (GGGGS)n, (EAAAK)n, etc.

In some examples described herein the extracellular portion of ACE2 may correspond to the wildtype extracellular fragment of ACE2; however extracellular fragments of ACE2 that have been modified by one or more modifications (mutations) can be used as the extracellular ACE2 fragment, including mutations are designed to improve binding to virus (e.g. SARS-CoV-2) or to eliminate the innate catalytic activity of the ACE2 enzyme. The extracellular fragment of ACE2 may exclude the collectrin domain (corresponding to amino acids 615-710 of the wild time human ACE2). In addition, any appropriate Fc domain can be used, including antibody Fc from different IgG isotypes (e.g. IgG3, IgG4), as well as Fc engineered to have different effector functions (e.g., LALA-PG to suppress Fcg-R binding, or YTE or LS mutations to improve FcRn binding, etc.). Any appropriate linker region may be used. For example, the linker region may be (GGGGS)ₙ for one or both linker regions (linking each of the two or more coronavirus binding/decoy domains to the Fc domain). In some examples, n for each flexible linker is between 1 and 26, and in particular, where n is between 2-25, between 3-24, between 4-22, between 5-20, between 6-20, between 3-10, between 4-15, etc.), or (EAAK)ₙ (where n is between 0 and 26, and in particular, where n is between 2-25, between 3-24, between 4-22, between 5-20, between 6-20, between 3-10, between 4-15, etc.). The lengths of the flexible linkers may be selected so that the average spacing between the two (or more) coronavirus binding/decoy domains is greater than about 14 nm in total (e.g., each linker may be about 5 nm or more).

Soluble angiotensin-converting enzyme 2 (ACE2) can act as a decoy molecule that can neutralize severe acute respiratory syndrome coronavirus 2 (SARS-CoV-2) by blocking the spike (S) protein of the viruses from binding ACE2 on host cells. Based on structure of ACE2 and S proteins, ACE2-Fc conjugates were engineered as described herein and included an extracellular segment of ACE2, in some examples without the C-terminal collectrin domain, and linked to human Ig domain IgG1-Fc) via an extended flexible linker that can enable improved bivalent binding of the molecule to S proteins on the virus.

This family of molecules, referred to herein as bivalent and flexibly linked ACE2-Fc decoys (or simply “flexibly linked. ACE2 decoys” for short) exhibit substantially greater binding affinity and neutralization potency than expected and this binding affinity. Interestingly, the neutralization potency of these flexibly linked ACE2 decoys is greater than that of full length ACE2-Fc decoys that do not include a flexible linker region, or that include a short linker region. These flexibly linked ACE2 decoys exhibited picomolar binding affinity (250 pM) and neutralization potency (IC50: 50 ng/mL). The flexibly linked ACE2 decoys also enabled effective trapping of fluorescent SARS-CoV-2 virus like particles in fresh human airway mucus, and can be stably nebulized using a commercial vibrating mesh nebulizer. Intranasal dosing of flexibly linked ACE2 decoys in hamsters as late as 2 days post-infection provided a 10-fold reduction in viral load in the nasal turbinate tissues by Day 4. These results strongly support the use of flexibly linked ACE2 decoys for inhaled immunotherapy of COVID-19 as well as other emerging viruses that use ACE2 as entry receptor.

One, non-limiting example of a flexibly linked ACE2 decoy is referred to as ACE2-(G4S)6-Fc, which includes two ACE2 extracellular domains (each excluding the C-terminal collectrin domain) that are flexibly linked via (GGGGS)₆ to an Fc domain. Although this particular ACE2-(G4S)6-Fc example is described in many of the examples and illustrations used herein, it should be understood other flexibly linked ACE2 decoys have been identified and shown to have similar properties. In general, the flexibly linked ACE2 decoys including two ACE2 extracellular domains with one or more mutations (see, e.g., table 1, described in greater detail below) that are each linked by a flexible linker having a length of greater than about 5 nm to an Fc domain may work as described herein and may share similar affinity and properties with ACE2-(G4S)₆-Fc.

Also described herein are engineered flexibly linked multi-valent bispecific) binding proteins for ACE2-targeted viruses that include only a single ACE2, but instead use one or more coronavirus-binding proteins, such as an antibody fragment with binding activity against ACE2-targeted viruses.

Any of the binding proteins (e.g., flexibly linked ACE2 decoys) described herein may be glycosylated (or selected for enrichment of glycosylation) of G0F glycosylation to which may enhance its muco-trapping potency. Increasing G0F content may improve trapping potency, e.g., by increasing G0F content to at least 15%, at least 20%, at least 25%, at least 30%, at least 35%, at least 40%, at least 45%, at least 50%, at least 55%, at least 60%, at least 65%, at least 70%, at least 80%, at least 90%, at least 95%, etc.

For example, described herein are isolated binding proteins that binds to ACE2-targeted viruses having an amino acid sequence comprising:

A-(B)ₙ-C (Formula I)

wherein: A is an extracellular portion of angiotensin-converting enzyme 2 (ACE2) excluding the collectrin domain, or a variant thereof; n is 1, 2, 3, 4, 5, 6, 7, 8, 9, 10, 11, 12, 13, 14, 15, 16, 17, 18, 19, 20, 21, 22, 23, 24 or 25; B is a polypeptide flexible linker; C is a fragment crystallization (Fc) domain, wherein the isolated binding protein dimerizes.

ACE2-targeted virus includes coronaviruses, such as SARS-like coronaviruses (e.g., SARS-CoV and SARS-CoV-2, SARS-CoV-1, NL63 seasonal coronavirus).

The binding proteins described herein may include a flexible linker if length sufficient so that the distance between the A domains of the dimers is greater than about 14 nm (e.g., greater than about 15 nm, greater than about 16 nm, greater than about 17 nm, greater than about 18 nm, greater than about 19 nm, greater than about 20 nm, etc.). The distance that the linker(s) in the dirtier may be determined stochastically and/or computationally; distance may refer to an average distance, as would be understood by those of skill in the art. Although the length of the flexible linkers may vary as the molecule configuration in space changes, below the minimum length (e.g., 14 nm, 15 nm, 16 nm, etc.) the percentage of binding proteins able to divalently on the target (e.g., spike proteins on an ACE2-targeted virus) may be below a threshold for efficacy.

For example, the length of the flexible polypeptide linker may be determined based on the number of residues of the polypeptide. For example, the number of residues may be 24 or greater, 25 or greater, 26 or greater, 27 or greater, 28 or greater, 20 or greater, 30 or greater, 31 or greater, 32 or greater, 33 or greater, 34 or greater, 35 or greater, 36 or greater, 37 or greater, 38 or greater, etc.

In general, the binding proteins described herein may include any appropriate Fc domain (e.g., of any of claims 1-2, wherein the Fc domain is a human IgA, WA or IgG Fc domain. The Fc domain may be a human IgG1 Fc domain. The Fc domain may comprise a YTE mutation, an LS mutation, or a LALA-PG mutation, or other modification to improve function.

In general, the extracellular portion of ACE2 may be an extracellular portion of a human ACE2, excluding the collectrin domain. The extracellular sequence may generally correspond to the sequence of the wildtype human ACE2 extracellular domain, e.g., a stretch of at least 40% (at least 45%, at least 50%, at least 55%, at least 60%, at least 65%, at least 70%, at least 75%, at least 80%, at least 85%, etc.) of the amino acid sequence from residues 18-614. In some examples the extracellular portion of ACE2 has an amino acid sequence identity of 80% or greater with the amino acid sequence of SEQ ID NO: 11. For example, the extracellular portion of ACE2 may have an amino acid sequence that has up to 10 amino acid difference within the amino acid of SEQ ID NO: 11. For example, the extracellular portion of ACE2 may include at least one mutation, or in some examples two or more mutations. The mutations may be at any of the positions identified in table 1 of FIGS. 16A-16B.

The polypeptide flexible linker may have any appropriate sequence. For example, the flexible linker may be a sequence of GGS, EGGS, GGGGS, etc. The sequence length (n) may be a minimum of, e.g., 2, 3, 4, 5, 6, 7, 8, 9, 10, etc., based on the length of the linker region, as described above. For example, if the flexible linker has a sequence of GGGGS, n may be 5 or greater (e.g., 6 or greater, 7 or greater, etc.). In some examples the binding protein includes the sequence of SEQ ID NO: 2 and SEQ ID NO: 4. In some examples, the binding protein includes the sequence of SEQ ID NO: 11 and SEQ ID NO: 4. In some examples the binding protein includes SEQ ID NO: 2 or SEQ ID NO: 11, a flexible linker such as (GGGS)n, and SEQ ID NO: 12 or SEQ ID NO: 13, where n is between 5 and 10 (e.g., n=6). Any of these binding proteins may include a hinge between the flexible linker and the Fc domain.

In general, any of these binding proteins may include an oligosaccharide having a G0 glycosylation pattern on the Fc domain. For example, the Fc domain may include an oligosaccharide having a G0 glycosylation pattern comprising a biantennary core glycan structure of Manα1-6(Manα-3)Manβ1-4GlcNAcβ1-4GlcNAcβ1 with terminal N-acetylglucosamine on each branch that enhances the trapping potency of the binding protein in mucus.

In general, the binding protein may be part of a mixture in which all or some (e.g., 20% or more, 30% or more, 40% or more, 50% or more, 60% or more, 70% or more, 80% or more, 90% or more, etc.) of the binding proteins are glycosylated and include the G0 glycosylation pattern on the Fc domain.

Thus, described are pharmaceutical composition comprising any of the binding protein and a pharmaceutically acceptable excipient. For example, the excipient, diluent, or carrier may be configured for inhalation. The composition may be configured for one or more of: oral, parenteral, intraperitoneal, transmucosal, transdermal, rectal, inhalable, and topical administration.

Also described herein are methods of treating a subject suffering from SARS-CoV-2, the method comprising administering a pharmaceutically acceptable amount of the pharmaceutical composition of any of these binding proteins. Administering may include applying the pharmaceutical composition systemically to the patient. In some examples, administering comprises applying the pharmaceutical composition to the patient's mucus membrane. Administering may include nebulizing the pharmaceutical composition.

For example, described herein are methods of treating or inhibiting a viral infection by an ACE2-targeted virus, the method comprising administering to the subject, via, an inhaled route, a binding protein of any of the binding proteins (e.g., any of the flexibly linked ACE2 decoys described herein). As mentioned, the ACE2-targeted virus may be SARS-CoV-2.

Also described herein are isolated binding proteins that binds to ACE2-targeted viruses having an amino acid sequence comprising:

A-(B)ₙ-C (Formula I)

wherein: A is an extracellular portion of angiotensin-converting enzyme 2 (ACE2) excluding the collectrin domain, having an amino acid sequence identity of 80% or greater with the amino acid sequence of SEQ ID NO: 11; n is 1, 2, 3, 4, 5, 6, 7, 8, 9, 10, 11, 12, 13, 14, 15, 16, 17, 18, 19, 20, 21, 22, 23, 24 or 25; B is a polypeptide flexible linker; C is a fragment crystallization (Fe) domain, wherein the isolated binding protein dimerizes, further wherein n is selected such that the distance between the A domains of the dimers is greater than 14 nm.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A shows one example of a 3D molecular structure of one example of a binding protein against SARS-Like Coronavirus comprising a dimer of ACE2-Fc, each monomer with a flexible linker, shown in this example as (GGGGS)n.

FIG. 1B shows an example of a dimer of ACE2-Fc without flexible linker.

FIGS. 2A-2B illustrates docking of different ACE2-Fc constructs (dimers) on S protein trimer, showing differences in “intra-spike” binding to S-protein. FIG. 2A shows an ACE2-Fc without a flexible linker can only bind mono-valently to the same S protein spike, as the geometry of this example of ACE2-Fc does not allow for the second Fab to bend and reach around to either of the two remaining available S-proteins on the S-protein trimer. FIG. 2B shows an example of an ACE2-Fc with flexible linkers (a flexibly linked ACE2 decoy) that allows for bivalent binding of a single ACE2-Fc molecule on a S protein trimer, e.g., when the linker is at least 5.7 nm.

FIG. 3 shows an example of a dimer of ACE2-Fc without a flexible linker, illustrating that it can potentially bind to two different spikes (i.e. “inter-spike” binding), but with limited frequency. The distance between the binding interface of the ACE2 domains is approximately 14.6 nm, which roughly equates to the inter-spike distance (approximately 14 to 15 nm) on the COVID19 virus surface when the S-proteins are vertically aligned. Due to lack of rotational flexibility on the ACE2 Fabs, it is likely that the two S trimer spikes would need to be substantially closer than the 15 nm distance in order for the ACE2-Fc with no flexible linkers to bind bivalently.

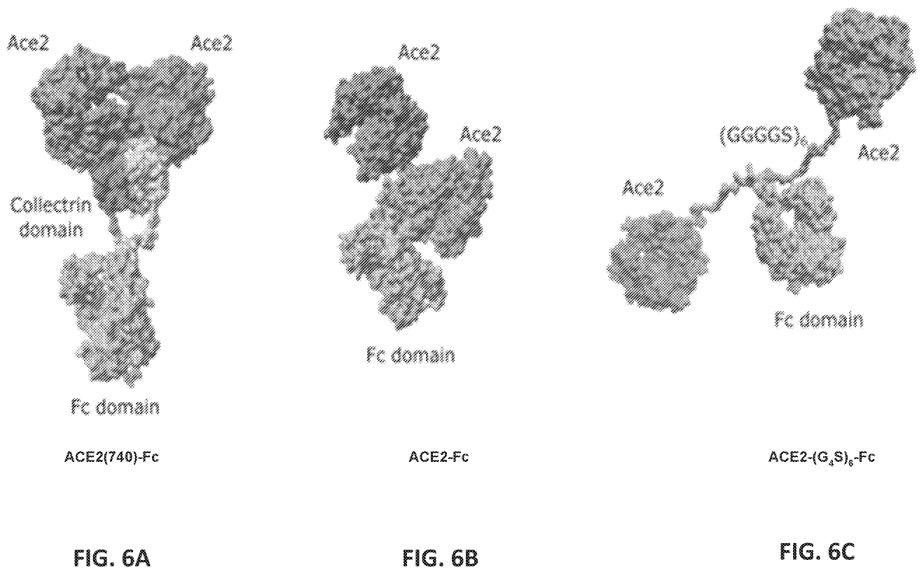

FIG. 4 illustrates a dimer of ACE2-Fc with a flexible linker (a flexibly linked ACE2 decoy) that can more readily achieve bivalent binding to two different S protein trimers. A linker length of 5.6 nm for both linkers makes it possible for two ACE2 domains to bind S proteins separated by 15 nm even when both S-trimers are vertically aligned as would naturally occur on the surface of the virus.

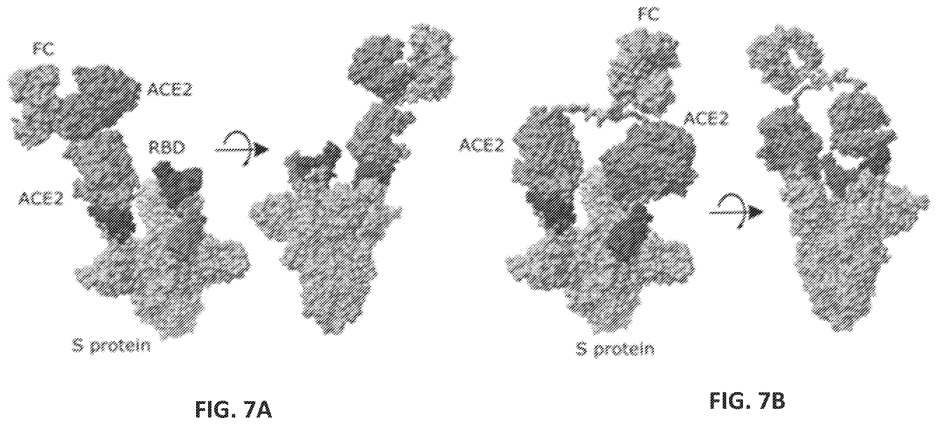

FIG. 5 shows an example of a proposed bispecific monoclonal antibody derived from CR3022 IgG (antibody to Human coronavirus SARS-CoV-2 Spike Glycoprotein 5) and ACE2 that can achieve bivalent binding to just one of the three trimeric S proteins on each S-protein spike of COVID19 without hindering each other. The N-terminus of RC3022 and C-terminus are separated by 9.8 nm, which could be bridged with a (GGGGS)₆ tinker.

FIGS. 6A-6C illustrate computational predictions of hypothetical structures of (linters of different ACE2 fusion proteins are shown. FIG. 6A shows an example in which an ACE2-Fc fusion comprised of the entire extracellular ACE2 molecule, including the collectrin domain, is linked to IgG1-Fc (referred to herein as ACE2(740)-Fc). As shown the ACE2 domains aggregate even when linked through the Fc domain) in this example. FIG. 6B shows an example in which the ACE2-Fc fusion includes the extracellular domain of ACE2 without collectrin domain, but linked to the Fc domain without a flexible linker. This example is referred to as ACE2-Fc. FIG. 6C shows an example of a flexibly linked ACE2 decoy in which two ACE2 fragments without the collectrin domain are linked to human IgG1-Fc via a 30 amino acid glycine-serine flexible linker (e.g., ACE2-(G4S)6-Fc).

FIG. 7A illustrate examples of computational predictions for the binding proteins shown in FIG. 6B (ACE2-Fc, without a flexible linker) and FIG. 6C (ACE2-(G4S)6-Fc). As shown in FIG. 7A, the computational prediction for ACE2-Fc shows that the ACE2-Fc will dock onto the S protein with only a single RBD domain. In contrast, as shown in FIG. 7B, ACE2-(G₄S)6-Fc (a flexibly linked ACE2 decoy) is predicted to dock on the S protein with two of the three RBD domains in the “up” position. FIG. 7C shows a Native-PAGE of ACE2-Fc (lane 2) and ACE2-(G₄S)6-Fc (lane 3). FIG. 7D is a size exclusion chromatography of ACE2-(G₄S)₆-Fc and ACE2-Fc. Both elution time and size are as expected. For the slightly larger ACE2-(G₄S)₆-Fc flexibly linked ACE2 decoy.

FIGS. 8A-8D illustrate the significantly different binding affinities of the example ACE fusion proteins shown in FIG. 6, as evaluated by SARS-CoV-2 S-protein ELISAs. FIG. 8A show representative concentration-dependent binding curves for ACE2-(G₄S)₆-Fc (black circle), ACE2-Fc (light gray square) and full length ACE2 decoy ACE2(740)-Fc (gray triangle). FIG. 8B shows ELISA-derived EC₅₀ values for different unique batches of the ACE2 fusion proteins of FIG. 6 (the same labels as in FIG. 8A apply). CH denotes ACE2-(G₄S)₆-Fc produced in CHO cells. FIG. 8C shows representative concentration-dependent binding curves for ACE2-(G₄S)₆-Fc against S proteins derived from different strains of virus, including WT (USA-WA1/2020), UK (B.1.1.7) and SA (B.1.351) strains; FIG. 8D shows EC50 data from the same set of strains.

FIGS. 9A-9C illustrate pseudovirus-based neutralization potency of the three different ACE2 fusion proteins shown in FIGS. 6A-6C above. In FIG. 9A, representative infectivity curves of pseudotyped SARS-CoV-2 virus across different concentrations of ACE2-decoys are shown. FIG. 9B shows IC50 data for each of the three categories of ACE2 bivalent fusion proteins, and FIG. 9C shows IC90 values estimated from the binding curves. Each data point represents independent experiments. There is a significant difference between the flexibly linked ACE2 decoy and the other fusion proteins.

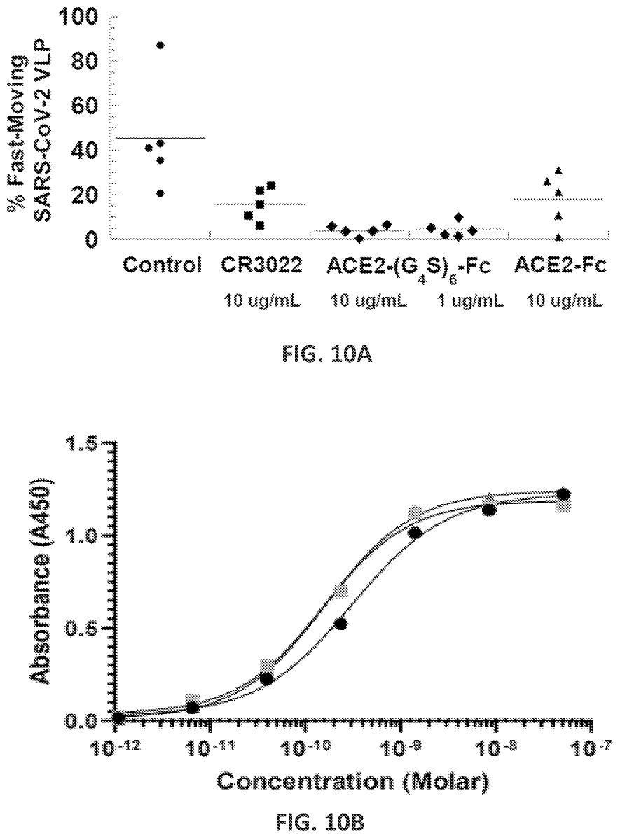

FIGS. 10A-JOB illustrate the effectiveness of flexibly linked. ACE2 decoys in mucotrapping. FIG. 10A shows a comparison of percent fast-moving SARS-CoV-2 showing that ACE2-(G₄S)₆-Fc effectively traps SARS-2 VLP in human AM with much greater potency than ACE2-Fc or CR3022 (CR3022 is a control anti-SARS-CoV-2 mAb). FIG. 10B shows the binding affinity of nebulized ACE2-(G₄S)₆-Fc evaluated by SARS-CoV-2 S-protein ELISAs. ACE2-(G₄S)₆-Fc collected from the upper chamber (full circle) and lower chamber (grey square) are compared to non-nebulized protein (triangle).

FIG. 11 illustrates a PCR-based assay for viral load in nasal turbinate tissues of SARS-CoV-2-infected hamsters collected at 4 days post infection,

FIGS. 12A-12B illustrate the biophysical characterization of nebulized ACE2-(G₄S)₆-Fc. FIG. 12C shows an example of a native-PAGE of nebulized ACE2-(G4S)₆-Fc, Samples were collected from the upper chamber (lanes 2, 5, 8), lower chamber (lanes 3, 6, 9), and left-over liquid (“dead volume”) after nebulization (lane 4, 7, 10) of the nebulization device. Data is shown for 3 repeats. FIG. 12B is a size exclusion chromatography of ACE2-(G₄S)₆-Fc including samples from before nebulization, samples collected from the upper chamber, lower chamber, or left-over liquid of the nebulization apparatus. Data representative of 3 repeats is shown.

FIG. 13 shows an example of the yield of the ACE2-Fc fusion protein as compared with the ACE2-(G₄S)₆-Fc (flexibly linked ACE2 decoy) after protein A affinity chromatography. Proteins were purified from 500 mL cultures of Expi293T cells.

FIG. 14 is an example showing differential Scanning fluorimetry of ACE2-(G₄S)₆-Fc. Data for three independent repeats is shown in the figure.

FIG. 15 shows the sequence of full-length ACE2 (human).

FIGS. 16A-16B shows table 1, illustrating the mutations to the full-length ACE2 polypeptide that may be made in any of the flexibly-linked ACE2 decoys described herein.

### DETAILED DESCRIPTION

In general, described herein are methods and compositions (e.g., engineered binding proteins) for binding to one or more ACE2-targeted viruses (e.g., SARS-CoV and SARS-CoV-2). These binding proteins may be used for treatment, prevention and/or reduction of infection by SARS-like coronavirus. In some examples, these binding proteins may be used for enhancing agglutination, enchainment and/or muco-trapping of ACE2-targeted viruses, including reducing the fraction of ACE2-targeted viruses that could permeate through mucus.

For example, described herein are angiotensin-converting enzyme 2 (ACE2) Immunoglobulin (IgG) hybrid binding proteins (referred to herein as flexibly linked. ACE2 decoys), that dimerize and have picomolar affinity for SARS-like coronavirus, including, in particular, for SARS-CoV-2. These proteins may be engineered for “muco-trapping,” including enhanced muco-trapping by selecting specifically for binding proteins that are glycosylated the Fc domain of the binding protein. These binding proteins may be used to treat or prevent SARS-CoV (e.g., SARS-CoV-2) infection, for example, for topical immunotherapy against ACE2-targeted viruses including SARS-CoV-2. These molecules may generally be fusions of an extracellular portion(s) of ACE2 (e.g., a portion of soluble angiotensin-converting enzyme 2 excluding the collectrin domain) to an Fc portion using a flexible linker, such as but not limited to (GGGGS)n, (EAAAK)n, etc.

Also described herein are binding proteins that are polyvalent for ACE2-targeted viruses and may include two (or in some examples, more) coronavirus-binding regions that are each flexibly linked by a flexible polypeptide linker to an Fc domain. The linkers may be sufficiently long and flexible so that both coronavirus-binding regions can bind to target (e.g., spike proteins), simultaneously.

The binding proteins described herein may enhance agglutination, facilitate enchained growth and/or improving muco-trapping of the ACE2-targeted viruses (e.g., SARS-CoV-2) as described herein. These binding proteins may stop the penetration of SARS-like CoV through mucus by improving the agglutination potency, facilitating enchained growth of the target and/or enabling muco-trapping, and may prevent, limit and/or treat infection.

### Definitions

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

As used herein the term “administration” or “administering” as used herein refers to the introduction of a composition into a subject by a chosen route. Administration can be local or systemic. For example, if the chosen route is intravenous, the composition (such as a composition including a disclosed antibody) is administered by introducing the composition into a vein of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Unless the context indicates otherwise, it is specifically intended that the various features described herein can be used in any combination. Moreover, the present invention also contemplates that in some examples of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

The term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

The transitional phrase “consisting essentially of” means that the scope of a claim is to be interpreted to encompass the specified materials or steps recited in the claim, and those that do not materially affect the basic and novel characteristic(s) of the claimed invention. Any of the methods and compositions described herein may be partially or completely exclusive of other components (e.g., may “consist of” or may “consist essentially of”). In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein, the term “amino acid substitution” refers to the replacement of one amino acid in a polypeptide with a different amino acid or with no amino acid (i.e., a deletion) In some examples, an amino acid in a polypeptide is substituted with an amino acid from a homologous polypeptide, for example, and amino acid in a recombinant SARS-CoV or SARS-CoV-2 polypeptide can be substituted with the corresponding amino acid from a different SARS-CoV or SARS-CoV-2 strain.

As used herein, the term “antibody” refers to a binding protein that specifically binds and recognizes an antigen such as SARS-CoV or SARS-CoV-2 S protein or an antigenic fragment thereof. The term “antibody” is used herein in the broadest sense and encompasses various antibody structures, including but not limited to monoclonal antibodies, polyclonal antibodies, bispecific antibodies, multispecific antibodies, chimeric antibodies, recombinant antibodies, and antigen binding fragments thereof, so long as they exhibit the desired antigen-binding activity.

As used herein, the term monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. The modifier “monoclonal” indicates the character of the antibody as being obtained from a substantially homogeneous population of antibodies, and is not to be construed as requiring production of the antibody by any particular method. In some examples, a monoclonal antibody is an antibody produced by a single clone of B-lymphocytes or by a cell into which nucleic acid encoding the light and heavy variable regions of the antibody of a single antibody (or an antigen binding fragment thereof) have been transfected, or a progeny thereof. In some examples monoclonal antibodies are isolated from a subject. Monoclonal antibodies can have conservative amino acid substitutions which have substantially no effect on antigen binding or other immunoglobulin functions. Exemplary methods of production of monoclonal antibodies are known, for example, see Harlow & Lane, Antibodies, A Laboratory Manual, 2nd ed. Cold Spring Harbor Publications, New York (2013).)

Typically, an immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda and kappa. There are five main heavy chain classes (or isotypes) which determine the functional activity of an antibody molecule: NM, IgD, IgG. IgA and IgE.

Each heavy and light chain contains a constant region (or constant domain) and a variable region (or variable domain; see, e.g., Kindt et al. Kuby Immunology, 6th ed., W. H. Freeman and Co., page 91 (2007)) In several examples, the heavy and the light chain variable regions combine to specifically bind the antigen. In additional examples, only the heavy chain variable region is required. For example, naturally occurring camelid antibodies consisting of a heavy chain only are functional and stable in the absence of light chain (see, e.g., Flamers-Casterman et al., Nature, 363:446-448, 1993; Sheriff et al., Nat. Struct. Biol., 3:733-736, 1996). References to “V_{H}” or “VH” refer to the variable region of an antibody heavy chain, including that of an antigen binding fragment, such as Fv, scFv, dsFv or Fab. References to “V_{L}” or “VL” refer to the variable domain of an antibody light chain, including that of an Fv, scFv, dsFv or Fab.

Light and heavy chain variable regions contain a “framework” region interrupted by three hypervariable regions, also called “complementarity-determining regions” or “CDRs” (see, e.g., Kabat et al., Sequences of Proteins of Immunological interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (“Sequences of Proteins of Immunological Interest,” 5th Ed. Public Health Service, National Institutes of Health, Bethesda, Md., 1991; “Kabat” numbering scheme), Al-Lazikani et al., (JMB 273,927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27:55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the variable domain of the heavy chain of the antibody in which it is found, whereas a V_{L} CDR1 is the CDR1 from the variable domain of the light chain of the antibody in which it is found. Light chain CDRs are sometimes referred to as LCDR1, LCDR2, and LCDR3. Heavy chain CDRs are sometimes referred to as HCDR1, HCDR2, and HCDR3.

As used herein; the phrase, an “antigen binding fragment” refers to a portion of a full length antibody that retains the ability to specifically recognize the cognate antigen, as well as various combinations of such portions. Non-limiting examples of antigen binding fragments include Fv, Fab, Fab′, Fab′-SH, F(ab)₂, diabodies; linear antibodies; single-chain antibody molecules (e.g. scFv); and bispecific and multispecific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2.sup.nd Ed., Springer Press, 2010).

A single-chain antibody (scFv) is a genetically engineered molecule containing the V_{H} and V_{L} domains of one or more antibody(ies) linked by a suitable polypeptide linker as a genetically fused single chain molecule (see, for example, Bird et al., Science, 242:423-426, 1988; Huston et al., Proc. Natl. Acad. Sci., 85:5879-5883, 1988; Ahmad et al., Clin. Dev. Immunol., 2012, doi:10.1155/2012/980250; Marbry, IDrugs, 13:543-549, 2010). The intramolecular orientation of the V_{H}-domain and the V_{L}-domain in a scFv, is typically not decisive for scFvs. Thus, scFvs with both possible arrangements (V_{H}-domain-linker domain-V_{L}-domain; V_{L}-domain-linker domain-V_{H}-domain) may be used.

In a dsFv the heavy and light chain variable chains have been mutated to introduce a disulfide bond to stabilize the association of the chains. Diabodies also are included, which are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90:6444-6448, 1993; Poljak et al., Structure, 2:1121-1123, 1994).

Antibodies also include genetically engineered forms such as chimeric antibodies (such as humanized murine antibodies) and heteroconjugate antibodies (such as bispecific antibodies). See also, Pierce Catalog and Handbook, 1994-1995 (Pierce Chemical Co., Rockford, Ill.); Kuby, J., Immunology, 3rd Ed., W.H. Freeman & Co., New York, 1997.

Non-naturally occurring antibodies can be constructed using solid phase peptide synthesis, can be produced recombinantly, or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies, are well known to those skilled in the art (Winter and Harris, Immunol. Today 14:243-246 (1993); Ward et al., Nature 341:544-546 (1989); Harlow and Lane, supra, 1988; Hilyard et al., Protein Engineering: A practical approach (IRL Press 1992); Borrabeck, Antibody Engineering, 2d ed. (Oxford University Press 1995); each of which is incorporated herein by reference).

As used herein, the term “humanized” antibody or antigen binding fragment refers to a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one example, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

As used herein, the phrase, “chimeric antibody” as used herein refers to an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some examples, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1″ Ed. New York: Cold Spring Harbor Laboratory Press, 2004. Print.; Lonberg, Nat. Biotech., 23: 1117-1125, 2005; Lonenberg, Curr. Opin. Immunol., 20:450-459, 2008).

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

As used herein the term “antigen” refers to a compound, composition, or substance that can stimulate the production of antibodies or a T cell response in an animal, including compositions that are injected or absorbed into an animal. An antigen reacts with the products of specific humoral or cellular immunity, including those induced by heterologous antigens, such as the disclosed SAILS-CoV or SARS-CoV-2 antigens. Examples of antigens include, but are not limited to, polypeptides, peptides, lipids, polysaccharides, combinations thereof (such as glycopeptides) and nucleic acids containing antigenic determinants, such as those recognized by an immune cell.

The term “binding protein” as used herein refers to at least one protein comprising a binding capability to a defined target. The target can be one or more analytes, antigens, autoantigens, proteins, polypeptides, etc. In some aspects, the binding protein can comprise a fusion protein. In addition to and in other aspects, the binding proteins of the present disclosure can also include one or more other molecules such as, for example, one or more immunoglobulins or immunoglobulin fragments. In some aspects, the binding protein is an antibody or antibody binding fragment thereof.

The term “fusion protein” as used herein relates to a protein comprising at least a first protein joined genetically to at least a second protein. A fusion protein is created through joining of two or more genes that originally coded for separate proteins. Thus, a fusion protein may comprise a multimer of different or identical binding proteins which are expressed as a single, linear polypeptide. Such fusion proteins may further comprise additional domains that are not involved in binding of the target, such as but not limited to, for example, multimerization moieties, polypeptide tags, polypeptide linkers.

As used herein the term “conservative” when used in connection with amino acid substitutions refers to those substitutions that do not substantially affect or decrease a function of a protein, such as the ability of the protein to induce an immune response when administered to a subject. For example, in some examples, a recombinant SARS-CoV or SARS-CoV-2 S protein or S1 fragment can include up to 1, 2, 3, 4, 5, 6, 7, 8, 9, or up to 10 conservative substitutions compared to a corresponding native SARS-CoV or SARS-CoV-2 protein sequence and induce an immune response to SARS-CoV or SARS-CoV-2 S protein in a subject. The term conservative variation also includes the use of a substituted amino acid in place of an unsubstituted parent amino acid.

Furthermore, a person skilled in the art will recognize that individual substitutions, deletions or additions which alter, add or delete a single amino acid or a small percentage of amino acids (for instance less than 5%, in some examples less than 1%) in an encoded sequence are conservative variations Where the alterations result in the substitution of an amino acid with a chemically similar amino acid.

Conservative amino acid substitution tables providing functionally similar amino acids are well known to one of ordinary skill in the art. The following six groups are examples of amino acids that are considered to be conservative substitutions for one another:
1) Alanine (A), Serine (5), Threonine (T);
2) Aspartic acid (D), Glutamic acid (E);
3) Asparagine (N), Glutamine (Q);
4) Arginine (R), Lysine (K);
5) Isoleucine (I), Leucine (L). Methionine (M), Valine (V); and
6) Phenylalanine (F), Tyrosine (Y), Tryptophan (W).

Non-conservative substitutions are those that reduce an activity or function of protein, e.g., a SARS-CoV or SARS-CoV-2 S protein, such as the ability to induce an immune response when administered to a subject. For instance, if an amino acid residue is essential for a function of the protein, even an otherwise conservative substitution may disrupt that activity. Thus, a conservative substitution does not alter the basic function of a protein of interest.

As used herein the term “expression” refers to transcription or translation of a nucleic acid sequence. For example, a gene is expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. The term “expression” is used herein to denote either transcription or translation. Regulation of expression can include controls on transcription, translation, RNA transport and processing, degradation of intermediary molecules such as mRNA, or through activation, inactivation, compartmentalization or degradation of specific protein molecules after they are produced.

As used herein, the phrase “expression control sequences” refer to nucleic acid sequences that regulate the expression of a heterologous nucleic acid sequence to which it is operatively linked. Expression control sequences are operatively linked to a nucleic acid sequence when the expression control sequences control and regulate the transcription and; as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-511, 1987). For example, when cloning in bacterial systems, inducible promoters such as pi, of bacteriophage lambda, plac, perp, ptac (perp-lac; hybrid promoter) and the like may be used. In one example, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

As used herein, the phrase, “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein the term “heterologous” refers to originating from a different genetic source. A nucleic acid molecule that is heterologous to a cell originated from a genetic source other than the cell in which it is expressed. In one specific, non-limiting example, a heterologous nucleic acid molecule encoding a recombinant SARS-CoV or SARS-CoV-2 polypeptide or specific antibody is expressed in a cell, such as a mammalian cell. Methods for introducing a heterologous nucleic acid molecule in a cell or organism are well known in the art, for example transformation with a nucleic acid, including electroporation, lipofection, particle gun acceleration, and homologous recombination.

As used herein, the phrase “host cells” refers to cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

As used herein “IgA” refers to a polypeptide belonging to the class of antibodies that are substantially encoded by a recognized immunoglobulin alpha gene. In humans, this class or isotype comprises IgA₁ and IgA₂. IgA antibodies can exist as monomers, polymers (referred to as pIgA) of predominantly dimeric form, and secretory IgA. The constant chain of wild-type IgA contains an 18-amino-acid extension at its C-terminus called the tail piece (tp). Polymeric IgA is secreted by plasma cells with a 15-kDa peptide called the J chain linking two monomers of IgA through the conserved cysteine residue in the tail piece.

As used herein, “IgG” refers to a polypeptide belonging to the class or isotype of antibodies that are substantially encoded by a recognized immunoglobulin gamma gene. In humans, this class comprises IgG₁, IgG₂, IgG₃, and IgG₄.

As used herein, the term “isolated” refers to a biological component (such as a protein, for example a disclosed nucleic acid encoding such an antigen) has been substantially separated or purified away from other biological components, such as other biological components in which the component naturally occurs, such as other chromosomal and extrachromosomal DNA, RNA, and proteins. Proteins, peptides and nucleic acids that have been “isolated” include proteins purified by standard purification methods. The term also embraces proteins or peptides prepared by recombinant expression in a host cell as well as chemically synthesized proteins, peptides and nucleic acid molecules. Isolated does not require absolute purity, and can include protein, peptide, or nucleic acid molecules that are at least 50% isolated, such as at least 75%, 80%, 90%, 95%, 98%, 99%, or even 99.9% isolated.

As used herein, a “linker” is a bi-functional molecule that can be used to link two molecules into one contiguous molecule, for example, to link a carrier molecule to a polypeptide. Non-limiting examples of peptide linkers include glycine-serine linkers, such as a (GGGGS)ₙ linker (where n is 0, 1, 2, 3, 4, 5, 6, 7, 8, 9, 10, 11, 12, 13, 14, 15, 16, 17, 18, 19, 20, 21, 22, 23, 24, or 25).

As used herein, the terms “conjugating,” “joining,” “bonding,” or “linking” can refer to making two molecules into one contiguous molecule; for example, linking two polypeptides into one contiguous polypeptide, or covalently attaching a carrier molecule or other molecule to a polypeptide. The linkage can be either by chemical or recombinant means. “Chemical means” refers to a reaction, for example, between the polypeptide moiety and the carrier molecule such that there is a covalent bond formed between the two molecules to form one molecule.

As used herein, “Severe acute respiratory virus syndrome coronavirus” or “SARS-CoV” refers to a β-coronavirus which is positive-sense, single stranded RNA virus belonging to the subfamily Coronavirinae and causes severe respiratory syndrome in humans. SARS-CoV has the same structure proteins as three other known groups of coronaviruses: spike glycoprotein (S), membrane protein (M), envelope protein (E) and nucleocapsid protein (N). Coronavirus N protein is required for coronavirus RNA synthesis, and has RNA chaperone activity that may be involved in template switch.

SARS-CoV spike glycoprotein is 1255 amino acids long, with low (20-27 percent) amino acid similarity among other coronaviruses. Its carboxyl terminus (C-terminus) is comprised of the transmembrane region and the cytoplasmic tail. The extracellular domain of the SARS-CoV spike glycoprotein is comprised of two heptad repeat regions which are known as heptad repeat region 1 (HR1) and heptad repeat region 2.

SARS-CoV spike glycoprotein has two functional domains: S1 and S2. S1 is responsible for the binding with its receptor angiotensin-converting enzyme 2 (ACE2) on host cells and defines the host range of the virus. S2 is the transmembrane subunit that facilitates viral and cellular membrane fusion. Membrane fusion occurs when there is a conformational change in the HRs to form a fusion core. The HRs of the protein fold into coiled-coil structure-called the fusogenic state-causing the HR domains of the S protein to fold into a hairpin-like formation. This hairpin structure results in the cellular and viral membranes being pulled together and ultimately fusing.

Other known β-coronaviruses include SARS-CoV-2 and MERS-CoV, both of which lead to severe and potentially fatal respiratory tract infections. The genome sequence of SARS-CoV-2 is 96.2% identical to a bat CoV RaTG13 and 79.5% identical to SARS-CoV. The sequence of SARS-CoV-2 from a number of different samples has been described in a variety of publications, such as, for example, Lu et al., Lancet, 395:565-574 (February 2020) and https://www.ncbi.nlm.nih.gov/genbank/sars-cov-2-seqs/, the contents of each are herein incorporated by reference.

As used herein, the phrase “neutralizing antibody” refers to an antibody which reduces the infectious titer of an infectious agent by binding to a specific antigen on the infectious agent. In some examples the infectious agent is a virus. In some examples, an antibody that is specific for SARS-CoV or SARS-CoV-2 S protein neutralizes the infectious titer of SARS-CoV or SARS-CoV-2. A “broadly neutralizing antibody” is an antibody that binds to and inhibits the function of related antigens, such as antigens that share at least 85%, 90%, 95%, 96%, 97%, 98% or 99% identity antigenic surface of antigen. With regard to an antigen from a pathogen, such as a virus, the antibody can bind to and inhibit the function of an antigen from more than one class and/or subclass of the pathogen. For example, with regard to SARS-CoV or SARS-CoV-2, the antibody can bind to and inhibit the function of an antigen, such as SARS-CoV or SARS-CoV-2 S protein from more than one strain of SARS-CoV or SARS-CoV-2. In one example, broadly neutralizing antibodies to SARS-CoV or SARS-CoV-2 S protein are distinct from other antibodies to SARS-CoV or SARS-CoV-2 S protein in that they neutralize a high percentage of the many types of SARS-CoV or SARS-CoV-2 in circulation.

As used herein the phrase “nucleic acid” refers to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

As used herein, the term “nucleotide” refers to but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Conventional notation is used herein to describe nucleotide sequences: the left-hand end of a single-stranded nucleotide sequence is the 5 ′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

As used herein the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

A first sequence is an “antisense” with respect to a second sequence if a polynucleotide whose sequence is the first sequence specifically hybridizes with a polynucleotide whose sequence is the second sequence.

As used herein, the phrase “operably linked” refers to a first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

As used herein, the phrase “pharmaceutically acceptable carrier(s) refers to routine and conventional carriers known in the art such as those described in Remington's Pharmaceutical Sciences, by E. W. Martin, Mack Publishing Co., Easton, Pa., 19th Edition, 1995. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and buffering agents and the like, for example sodium acetate or sorbitan monolaurate. In particular aspects, suitable for administration to a subject the carrier may be sterile; and/or suspended or otherwise contained in a unit dosage form containing one or more measured doses of the composition suitable to induce the desired anti-SARS-CoV or SARS-CoV-2 immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

As used herein, the term “polypeptide” refers to any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Amino acids in a peptide, polypeptide or protein generally are chemically bound together via amide linkages (CONH). Additionally, amino acids may be bound together by other chemical bonds. For example, linkages for amino acids or amino acid analogs can include CH₂NH—, —CH₂S—, —CH₂—CH₂—CH═CH— (cis and trans), —COCH₂—CH(OH)CH₂—, and —CHH₂SO— (These and others can be found in Spatola, in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci pp. 463-468, 1980; Hudson, et al., Int J Pept Prot Res 14:177-185, 1979; Spatola et al. Life Sci 38:1243-1249, 1986; Harm J. Chem. Soc Perkin Trans. 1307-314, 1982; Almquist et al. J. Med. Chem. 23:1392-1398, 1980; Jennings-White et al. Tetrahedron Lett 23:2533; 1982; Holladay et al. Tetrahedron. Lett 24:4401-4404, 1983; and Hruby Life Sci 31:189-199, 1982.

As used herein, the term “sample” or “biological sample” refers to a biological specimen containing genomic DNA, RNA (including mRNA), protein, or combinations thereof, obtained from a subject. Examples include, but are not limited to, peripheral blood, tissue, cells, urine, saliva, tissue biopsy, fine needle aspirate, surgical specimen, and autopsy material.

As used herein, the term “sequence identity” refers to the similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs, orthologs, or variants of a polypeptide will possess a relatively high degree of sequence identity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Rio. 24:307-31, 1994, Altschul et al. J. Mol. Biol. 215:403-10, 1990, presents a detailed consideration of sequence alignment methods and homology calculations.

Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a peptide sequence that has 1166 matches when aligned with a test sequence having 1554 amino acids is 75.0 percent identical to the test sequence (1166/1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least about 75%, for example at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Sambrook et al. (Molecular Cloning: A Laboratory Manual, 4.sup.th ed. Cold Spring Harbor, N.Y., 2012) and Ausubel et al. (In Current Protocols in Molecular Biology, John Wiley & Sons, New York, through supplement 104, 2013). One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395, 1984.

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989). An oligonucleotide is a linear polynucleotide sequence of up to about 100 nucleotide bases in length.

As used herein, reference to “at least 80% identity” refers to “at least 80%, at least 85%, at least 90%, at least 91%, at least 92%, at least 93%, at least 94%, at least 95%, at least 96%, at least 97%, at least 98%, at least 99%, or even 100% identity” to a specified reference sequence.

As used herein, the phrase “signal peptide” refers to a short amino acid sequence (e.g., approximately 10-35 amino acids in length) that directs newly synthesized secretory or membrane proteins to and through membranes (for example, the endoplasmic reticulum membrane). Signal peptides are typically located at the N-terminus of a polypeptide and are removed by signal peptidases. Signal peptide sequences typically contain three common structural features: a N-terminal polar basic region (n-region), a hydrophobic core, and a hydrophilic c-region). Exemplary signal peptide sequences are set forth as SEQ ID NOS.: 1 and 6.

As used herein, the phrase “specifically bind(s)”, when referring to the formation of an antibody:antigen protein complex, or a protein:protein complex, refers to a binding reaction which determines the presence of a target protein, peptide, or polysaccharide (for example a glycoprotein), in the presence of a heterogeneous population of proteins and other biologics. Thus, under designated conditions, a particular antibody or protein binds preferentially to a particular target protein, peptide or polysaccharide (such as an antigen present on the surface of a pathogen, for example SARS-CoV or SARS-CoV-2 S protein) and does not bind in a significant amount to other proteins or polysaccharides present in the sample or subject. Specific binding can be determined by methods known in the art. A first protein or antibody “specifically binds to a target protein when the interaction has a K_{D} of less than about 10⁻⁶ molar, such as less than about 10⁻⁷ molar, less than about 10⁻⁸ molar, less than about 10⁻⁹ molar, less than about 10⁻¹⁰ molar, etc.

A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein. See Harlow & Lane, Antibodies, A Laboratory Manual, 2nd ed., Cold Spring Harbor Publications, New York (2013), for a description of immunoassay formats and conditions that can be used to determine specific immunoreactivity.

As used herein, the term “subject” refers to a living multi-cellular vertebrate organism, a category that includes human and non-human mammals. In an example, a subject is a human. In a particular example, the subject is a human or a camel, or a bat. In an additional example, a subject is selected that is in need of inhibiting of a SARS-CoV or SARS-CoV-2 infection. For example, the subject is either uninfected and at risk of SARS-CoV or SARS-CoV-2 infection or is infected and in need of treatment.

As used herein, the phrase, a “therapeutically effective amount” refers to the amount of agent, such as a disclosed antibody, that is sufficient to prevent, treat (including prophylaxis), reduce and/or ameliorate the symptoms and/or underlying causes of a disorder or disease, for example to prevent, inhibit, and/or treat SARS-CoV or SARS-CoV-2 infection. In some examples, a therapeutically effective amount is sufficient to reduce or eliminate a symptom of a disease, such as SARS-CoV or SARS-CoV-2 infection. For instance, this can be the amount necessary to inhibit or prevent viral replication or to measurably alter outward symptoms of the viral infection. In general, this amount will be sufficient to measurably inhibit virus replication or infectivity.

This description is not intended to be a detailed catalog of all the different ways in which the invention may be implemented, or all the features that may be added to the instant invention. For example, features illustrated with respect to one example may be incorporated into other examples, and features illustrated with respect to a particular example may be deleted from that example. In addition, numerous variations and additions to the various examples suggested herein will be apparent to those skilled in the art in light of the instant disclosure which do not depart from the instant invention. Hence, the following specification is intended to illustrate some particular examples of the invention, and not to exhaustively specify all permutations, combinations and variations thereof.

In one example, a desired response is to inhibit or reduce or prevent SARS-CoV or SARS-CoV-2 infection. The SARS-CoV or SARS-CoV-2 infected cells do not need to be completely eliminated or reduced or prevented for the composition to be effective. For example, administration of a therapeutically effective amount of the agent can decrease the number of SARS-CoV or SARS-CoV-2 infected cells (or prevent the infection of cells) by a desired amount, for example by at least 10%, at least 20%, at least 50%, at least 60%, at least 70%, at least 80%, at least 90%, at least 95%, at least 98%, or even at least 100% (elimination or prevention of detectable SARS-CoV or SARS-CoV-2 infected cells), as compared to the number of SARS-CoV or SARS-CoV-2 infected cells in the absence of the composition.

The therapeutically effective amount of a disclosed antibody can depend on the subject being treated, the severity and type of the condition being treated, and the manner of administration. A unit dosage form of the antibody can be packaged in a therapeutic amount, or in multiples of the therapeutic amount, for example, in a vial (e.g., with a pierceable lid) or syringe having sterile components.

Treating or preventing a disease: Inhibiting the full development of a disease or condition, for example, in a subject who is at risk for a disease such as SARS-CoV or SARS-CoV-2 infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

By the terms “treat,” “treating,” or “treatment of” (or grammatically equivalent terms) it is meant that the severity of the subject's condition is reduced or at least partially improved or ameliorated and/or that some alleviation, mitigation or decrease in at least one clinical symptom is achieved and/or there is a delay in the progression of the condition.

As used herein, the terms “prevent.” “prevents,” or “prevention” and “inhibit,” “inhibits,” or “inhibition” (and grammatical equivalents thereof) are not meant to imply complete abolition of disease and encompasses any type of prophylactic treatment that reduces the incidence of the condition, delays the onset of the condition, and/or reduces the symptoms associated with the condition after onset.

An “effective,” “prophylactically effective,” or “therapeutically effective” amount as used herein is an amount that is sufficient to provide some improvement or benefit to the subject. Alternatively stated, an “effective,” “prophylactically effective,” or “therapeutically effective” amount is an amount that will provide some delay, alleviation, mitigation, or decrease in at least one clinical symptom in the subject. Those skilled in the art will appreciate that the effects need not be complete or curative, as long as some benefit is provided to the subject.

The term “reduces” or “reduction” as used herein is a relative term, such that an agent reduces a response or condition if the response or condition is quantitatively diminished following administration of the agent, or if it is diminished following administration of the agent, as compared to a reference agent. Similarly, the term “prevents” does not necessarily mean that an agent completely eliminates the response or condition, so long as at least one characteristic of the response or condition is eliminated. Thus, a composition that reduces or prevents an infection or a response, can, but does not necessarily completely, eliminate such an infection or response, so long as the infection or response is measurably diminished, for example, by at least about 50%, such as by at least about 70%, or about 80%, or even by about 90% of (that is to 10% or less than) the infection or response in the absence of the agent, or in comparison to a reference agent.

As used herein, the term “vector” refers to a nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses. A replication deficient viral vector is a vector that requires complementation of one or more regions of the viral genome required for replication due to a deficiency in at least one replication-essential gene function. For example, such that the viral vector does not replicate in typical host cells, especially those in a human patient that could be infected by the viral vector in the course of a therapeutic method.

### Isolated Binding Proteins of Formula I

In one aspect, the present disclosure relates to an isolated binding protein that specifically binds to an epitope on a SARS-CoV and/or SARS-CoV-2 protein. Specifically, the isolated binding protein that specifically binds to an epitope on a SARS-CoV and/or SARS-CoV-2 protein can neutralize SARS-CoV and/or SARS-CoV-2 infection. Specifically, the isolated binding protein has an amino acid sequence comprising formula I:

A-(B)ₙ-C (Formula I)

wherein A is receptor utilized by a SARS-CoV and/or SARS-CoV-2 protein to mediate cellular entry, such as, for example, an angiotensin-converting enzyme 2 (ACE2), DPP4 or a variant thereof; n is 1, 2, 3, 4, 5, 6, 7, 8, 9, 10, 11, 12, 13, 14, 15, 16, 17, 18, 19, 20, 21, 22, 23, 24 or B is a polypeptide linker; and C is a fragment crystallization (Fe) domain. These proteins typically dimerize (e.g., through the Fc domain).

The Fc domain used in Formula I can be any a human Fc domain and can be a human IgA, IgM or IgG Fc domain. Additionally, the Fc domain can be an optimized Fc domain, such as that described in U.S. Patent Application No. 2010/093979. In one aspect of the present disclosure, the Fc domain is IgG₁. Additionally, the Fc domain can contain one or more amino acid substitutions (e.g., such as a conservative substitution) or mutations, such as, for example, to allow for enhanced Neonatal Fc-receptor (FcRn) binding.

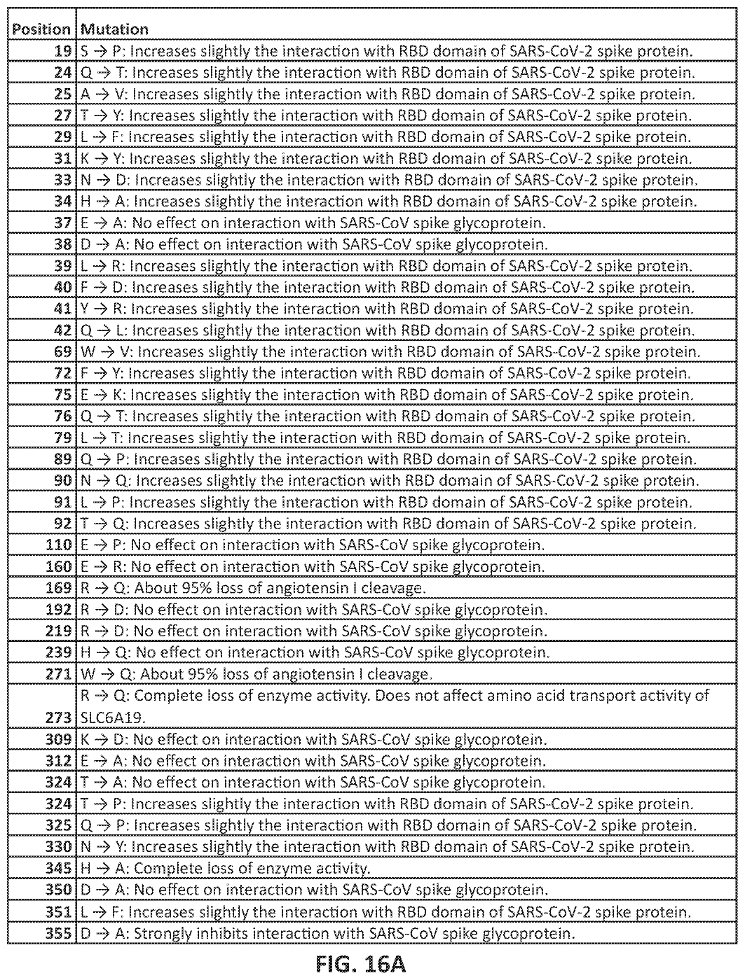

The ACE2 used in Formula I can be a human ACE2. The ACE2 or a fragment thereof (such fragments having a length of at least 10 amino acids, at least 15 amino acids, at least 20 amino acids, at least 25 amino acids, at least 30 amino acids, at least 40 amino acids, at least 45 amino acids, at least 50 amino acids, etc.) can be used in Formula I. In some aspects, the extracellular domain of human ACE2 or a fragment thereof is used. In particular, the extracellular domain excluding the collectrin domain. FIG. 15 shows an annotated sequence listing of wild type human ACE2. This sequence shows the collectrin domain, amino acids 615-740 (boxed). A portion of the extracellular region of the ACE2 protein (e.g., amino acids 17, 19 or 19 to amino acids 614, or a portion thereof, may be used as described herein. See, SEQ ID NO: 11.

In general, the composition and methods described for Formula I herein may be used with a peptide that is about 80% identical to the extracellular region of ACE2 or a portion thereof, excluding the collectrin domain. Additionally, the ACE2 used in Formula I can contain one or more amino acid substitutions (e.g., such as a conservative substitution) or mutations. In one aspect, the mutations eliminate the innate enzymatic activity of the ACE2 molecule while keeping/preserving the dimerization domain of the Fc domain. Examples of ACE2 sequences that can be used in the present disclosure are SEQ ID NOS.: 2, 4, 11, 15, 17, 19, 21, and 23 which provide the amino acid sequence of an ACE2 that contain two substitutions or mutations which can be used in the binding protein described herein. Both SEQ ID NOS. 2 and 4 contain a H374N and H378N substitutions or mutations.

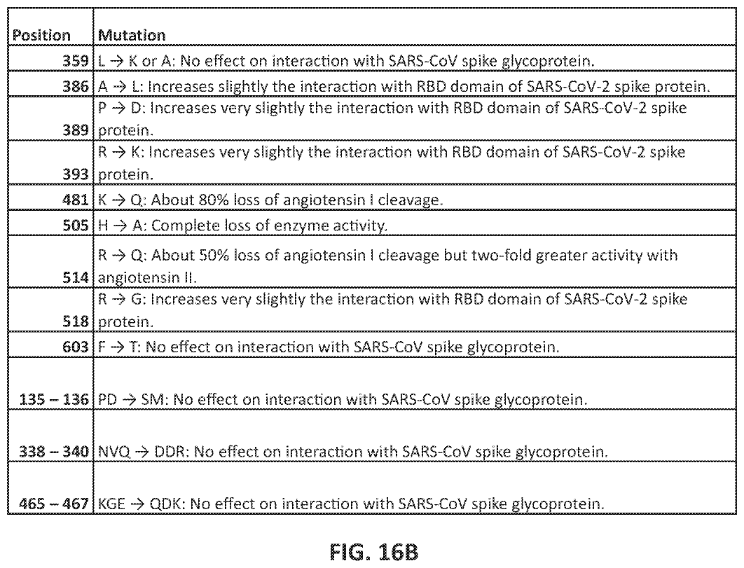

Table 1 of FIG. 16 illustrates amino acid mutations that may be made individually or collectively in the extracellular ACE2 polypeptide sequence. Specifically, one or more (or all) of the amino acids in these residues may be modified and the activity of the flexibly linked ACE2 decoys described herein may be preserved (and in some cases enhanced as compared to those formed by wild time extracellular ACE2 polypeptide without the collectrin domain). For example, one or more of the amino acids of residue positions 19, 20, 24, 25, 27, 29, 31, 33, 34, 35, 37, 38, 39, 40, 41, 42, 69, 72, 75, 76, 79, 89, 90, 91, 92, 101, 110, 135-136, 160, 169, 192, 219, 239, 271, 273, 309, 312, 324, 324, 325, 330, 338-340, 345, 350, 351, 355, 359, 386, 389, 393, 465-467, 481, 505, 514, 518, and/or 603. The particular change in these residues may be as indicated in Table 1, or they may be different; in some cases, the amino acid change may be a conservative change, e.g., based on charge and/or size. For example, SEQ ID NO:15 shows an example of an extracellular ACE2 polypeptide within the collectrin domain in which five residues are modified: residues K31F, N33D, H34S, E35Q, and H345L. This variant of ACE2 may be linked via an appropriate flexible linker as described herein to a Fc domain to form a flexibly linked ACE2 decoy, one example of which is shown in SEQ ID NO: 16. SEQ ID NO: 17 shows another example of a variant of ACE2 (extracellular ACE2 excluding collectrin domain and modifying residues T27Y, L79T, N330Y) that may be linked via an appropriate flexible linker as described herein to a Fc domain to form a flexibly linked ACE2 decoy, one example of which is shown in SEQ ID NO: 18. SEQ ID NO: 19 shows another example of a variant of ACE2 (extracellular ACE2 excluding collectrin domain and modifying residues T20I, H34A, T92Q, and Q101 H) that may be linked via an appropriate flexible linker as described herein to a. Fc domain to form a flexibly linked ACE2 decoy, one example of which is shown in SEQ ID NO: 20. SEQ ID NO: 21 shows another example of a variant of ACE2 (extracellular ACE2 excluding collectrin domain and modifying residues A25V, K31N, E34K and L79F) that may be linked via an appropriate flexible linker as described herein to a Fc domain to form a flexibly linked ACE2 decoy, one example of which is shown in SEQ TD NO: 22. SEQ TD NO: 23 shows another example of a variant of ACE2 (extracellular ACE2 modifying residue T27W) that may be linked via an appropriate flexible linker as described herein to a Fc domain to form a flexibly linked ACE2 decoy, one example of which is shown in SEQ ID NO: 24.

Any polypeptide linker, and particularly flexible, can be used in Formula I to link the extracellular ACE2 excluding the collectrin domain, to the Fc domain. In some examples, the linker has the sequence of GGGGS (SEQ ID NO:11).

In some examples, the binding protein can also comprise a hinge between the polypeptide linker and the Fc domain in Formula I. The location of the hinge in Formula I is not critical. The hinge region may be before the flexible linker (e.g., between the flexible linker and the extracellular ACE2 domain), within the flexible linker (e.g., (G4S)2-hinge-(G4S)4, etc.), or after (e.g., between the flexible linker and the Fc domain).

In some additional examples, the binding protein of Formula I can also contain a signal peptide. An example of a signal peptide that can be used is shown in SEQ ID NOS. 1 and 5. Other signal sequences may be used. The location of the signal peptide in Formula I is not critical.

In some examples, the binding protein is an antibody or antibody binding fragment thereof. When the binding protein is an antibody, the antibody can be a monoclonal antibody, humanized antibody, a recombinant antibody, a chimeric antibody, a human antibody, hi-specific antibody or a multi-specific antibody. When the binding protein is an antibody binding fragment it can be a single chain antibody, an Fab fragment, an F(ab′)2 fragment, an Fab′ fragment, an Fsc fragment, an Fv fragment, an scFv, an sc(Fv)2, or a diabody. Methods for making antibodies and antibody binding fragments are well known in the art.

In certain aspects, amino acid sequence variants of the binding protein provided herein are contemplated. For example, it may be desirable to improve the binding affinity and/or other biological properties of the binding protein (e.g., such as when the binding protein is an antibody). Amino acid sequence variants of a binding protein (e.g., antibody) may be prepared by introducing appropriate modifications into the nucleotide sequence encoding the binding protein (e.g., antibody), or by peptide synthesis. Such modifications include, for example, deletions from, and/or insertions into and/or substitutions of residues within the amino acid sequences of the binding protein. Any combination of deletion, insertion, and substitution can be made to arrive at the final construct, provided that the final construct possesses the desired characteristics, e.g., antigen-binding.

Examples of binding proteins of Formula I of the present disclosure include those shown in the figures, and discussed in the examples, below. The amino acid sequences for these binding proteins are provided in the sequence listing.

### Isolated BiSpecific Binding Proteins of Formula II

Also described herein are bispecific binding protein that specifically binds to at least one epitope on SARS-CoV and/or SARS-CoV-2 protein. Specifically, the isolated bispecific binding protein that specifically binds to at least one epitope on a SARS-CoV and/or SARS-CoV-2 protein can neutralize SARS-CoV and/or SARS-CoV-2 infection. Specifically, the isolated specific binding protein comprises at least one heavy chain variable region having an amino acid sequence comprising formula II:

X-(Y)ₙ-Z (Formula II)

wherein X is (i) is receptor utilized by a SARS-CoV and/or SARS-CoV-2 protein to mediate cellular entry, such as, for example, an angiotensin-converting enzyme 2 (ACE2), DPP4 or a variant thereof; or (ii) a variable heavy chain region from an antibody that binds to an epitope on SARS-CoV, SARS-CoV-2 or a fragment thereof; n is 1, 2, 3, 4, 5, 6, 7, 8, 9, 10, 11, 12, 13, 14, 15, 16, 17, 18, 19, 20, 21, 22, 23, 24 or 25; Y is a polypeptide linker; and Z is (i) is receptor utilized by a SARS-CoV and/or SARS-CoV-2 protein to mediate cellular entry, such as, for example, an angiotensin-converting enzyme 2 (ACE2), DPP4 or a variant thereof; or (ii) a variable heavy chain region from an antibody that binds to SARS-CoV, SARS-CoV-2 or a fragment thereof, provided that when (a) X is receptor utilized by a SARS-CoV and/or SARS-CoV-2 protein to mediate cellular entry, Z is a variable heavy chain region from an antibody that binds to SARS-CoV, SARS-CoV-2 or a fragment thereof; or (b) X is a variable heavy chain region from an antibody that binds to SARS-CoV, SARS-CoV-2 or a fragment thereof, Z is a receptor utilized by a SARS-CoV and/or SARS-CoV-2 protein to mediate cellular entry. In Formula II, if n is 0, no polypeptide linker is present.

The ACE2 used in Formula II can be a human ACE2, or variants (as already described above). A full length ACE2 or a fragment thereof (such fragments having a length of at least 10 amino acids, at least 15 amino acids, at least 20 amino acids, at least 25 amino acids, at least 30 amino acids, at least 40 amino acids, at least 45 amino acids, at least 50 amino acids, etc.) can be used in Formula II. In some aspects, the extracellular domain of human ACE2 or a fragment thereof can be used. Additionally, the ACE2 used in Formula II can contain one or more amino acid substitutions (e.g., such as a conservative substitution) or mutations. In one aspect, the mutations eliminate the innate enzymatic activity of the ACE2 molecule while keeping/preserving the dimerization domain of the Fc domain. Examples of ACE2 sequences that can be used in the present disclosure are SEQ ID NOS.: 2, 4, 11, 15, 17, 19, 21, and 23 which provide the amino acid sequence of an ACE2 that contain two substitutions or mutations which can be used in the binding protein described herein. Both SEQ ID NOS, 2 and 4 contain a H374N and H378N substitutions or mutations.

Any polypeptide linker can be used in Formula II to link the X to Z in Formula II. In some examples, the linker has the sequence of GGGGS (SEQ ID NO:11). Alternatively, in some examples, no linker is present and X is directly connected to Z (e.g., when n is 0).

In some examples, the binding protein can also comprise a hinge between the polypeptide linker and X and Z in Formula II. The location of the hinge in Formula II is not critical.

In some additional examples, the binding protein of Formula II can also contain a signal peptide. An example of a signal peptide that can be used is shown in SEQ ID NOS. 1 and 5. The location of the single peptide in Formula II is not critical.

As mentioned previously, in Formula II, if X is ACE2 then Z is a variable heavy chain region from a binding protein that specifically binds at least one epitope on SARS-CoV, SARS-CoV-2 or a fragment thereof (e.g., a fragment of SARS-CoV or a fragment of SARS-CoV-2). Alternatively, if X is a variable heavy chain region from a binding protein that specifically binds at least one epitope on SARS-CoV, SARS-CoV-2 or a fragment thereof, then Z is ACE2. Examples of a variable heavy chain region from a binding protein that specifically binds at least one epitope on SARS-CoV-2 that can be used in the binding protein is monoclonal antibody CR3014 or CR3022, which is described in J. ter Meulen, PLoS Medicine, 3(7):1071-1079 (July 2006), the contents of which are herein incorporated by reference. The entire variable heavy chain region from a binding protein (such as CR3014 or CR3022) that specifically binds at least one epitope on SARS-CoV or SARS-CoV-2 can be used, or a fragment thereof (such fragments having a length of at least 10 amino acids, at least 15 amino acids, at least 20 amino acids, at least 25 amino acids, at least 30 amino acids, at least 40 amino acids, at least 45 amino acids, at least 50 amino acids, at least 60 amino acids, at least 70 amino acids, at least 80 amino acids, at least 90 amino acids, at least 100 amino acids, etc.)

In some examples, the binding protein is an antibody or antibody binding fragment thereof. Wharf the binding protein is an antibody, the antibody can be a hi-specific antibody or a multi-specific antibody. In some aspects, the bispecific antibody can be a scFv. Methods for making antibodies and antibody binding fragments are well known in the art.

In certain aspects, amino acid sequence variants of the binding protein provided herein are contemplated. For example, it may be desirable to improve the binding affinity and/or other biological properties of the binding protein (e.g., such as when the binding protein is an antibody). Amino acid sequence variants of a binding protein (e.g, antibody) may be prepared by introducing appropriate modifications into the nucleotide sequence encoding the binding protein (e.g., antibody), or by peptide synthesis. Such modifications include, for example, deletions from, and/or insertions into and/or substitutions of residues within the amino acid sequences of the binding protein. Any combination of deletion, insertion, and substitution can be made to arrive at the final construct, provided that the final construct possesses the desired characteristics, e.g., antigen-binding.

The bispecific binding proteins of Formula II can also include other proteins such as variable light chain regions from other antibodies, variable heavy chains regions from other antibodies, one or more CDRs, one or more light and heavy chain constant regions, framework regions and Fc domains from other binding proteins. If an Fc domain is used, the Fc domain can be any a human Fc domain such as a human IgA, IgM or IgG Fc domain. Additionally, the Fc domain can be an optimized Fc domain, such as that described in U.S. Patent Application No. 2010/093979. In one aspect of the present disclosure, the Fc domain is Ig Additionally, the Fc domain can contain one or more amino acid substitutions (e.g., such as a conservative substitution) or mutations, such as, for example, to allow for enhanced Neonatal Fc-receptor (FcRn) binding. An example of such other proteins include one or more variable light chain regions from antibodies that specifically bind to at least one epitope on SARS-CoV and/or SARS-CoV-2. For example, the light chain variable region of CR3022 having the amino acid sequence in SEQ ID NO:6 can be used with the binding proteins of Formula II to make bispecific antibodies.

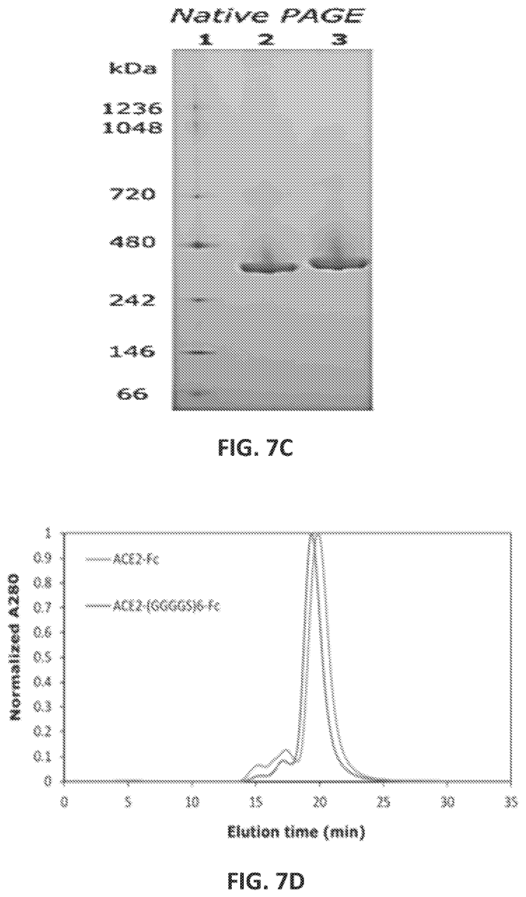

An example of a bispecific; binding protein of Formula II of the present disclosure is shown in FIG. 5. The amino acid sequence for this bispecific binding protein is provided in the sequence listing.

### Polynucleotides and Expression

Polynucleotides encoding a binding protein of Formula I or II that specifically binds ace epitope on a SARS-CoV and/or SARS-CoV-2 protein are also provided. These polynucleotides include DNA, cDNA and RNA sequences which encode the disclosed binding protein of Formula I or II. Nucleic acids encoding these molecules can readily be produced by one of skill in the art, using the amino acid sequences provided herein (such as the CDR and heavy chain and light chain sequences for production of antibodies), sequences available in the art (such as framework sequences), and the genetic code. One of skill in the art can readily use the genetic code to construct a variety of functionally equivalent nucleic acids, such as nucleic acids which differ in sequence but Which encode the same antibody sequence, or encode a conjugate or fusion protein including the nucleic acid sequence.

Polynucleotides encoding the disclosed binding protein of Formula I or II can be prepared by any suitable method including, for example, cloning of appropriate sequences or by direct chemical synthesis by methods such as the phosphodiester method of Narang et at, Meth. Enzymol. 68:90-99; 1979; the phosphodiester method of Brown et at, Meth. Enzymol. 68:109-151, 1979; the diethylphosphoramidite method of Beaucage et at, Tetra. Lett. 22:1859-4862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence or by polymerization with a DNA polymerase using the single strand as a template. One of skill would recognize that while chemical synthesis of DNA is generally limited to sequences of about 100 bases, longer sequences may be obtained by the ligation of shorter sequences.

Examples of appropriate cloning and sequencing techniques, and instructions sufficient to direct persons of skill through many cloning exercises are known (see, e.g. Sambrook et al. (Molecular Cloning: A Laboratory Manual, 4th ed, Cold Spring Harbor, N.Y., 2012) and Ausubel et al. (In Current Protocols in Molecular Biology, John Wiley & Sons, New York, through supplement 104, 2013). Product information from manufacturers of biological reagents and experimental equipment also provide useful information. Such manufacturers include the SIGMA Chemical Company (Saint Louis, Mo.), R&D Systems (Minneapolis, Minn.), Pharmacia Amersham (Piscataway, CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (Carlsbad, Calif.), and Applied Biosystems (Foster City, Calif.); as well as many other commercial sources known to one of skill.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

The nucleic acid molecules can be expressed in a recombinantly engineered cell such as bacteria, plant, yeast, insect and mammalian cells. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, e.g., Helgason and Miller (Eds.), 2012, Basic Cell Culture Protocols (Methods in Molecular Biology), 4th Ed., Humana. Press). Examples of commonly used mammalian host cell lines are VERO and HeLa cells, CHO cells, and WI38, BHK, and COS cell lines, although cell lines may be used, such as cells designed to provide higher expression, desirable glycosylation patterns, or other features. In some examples, the host cells include HEK293 cells or derivatives thereof, such as GnTI⁻/− cells (ATCC® No. CRL-3022), or HEK-293F cells.

The expression of nucleic acids encoding the proteins described herein can be achieved by operably linking the DNA or cDNA to a promoter (which is either constitutive or inducible), followed by incorporation into an expression cassette. The promoter can be any promoter of interest, including a cytomegalovirus promoter and a human T cell lymphotrophic virus promoter (HTLV)-1. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signal for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, at the minimum, a strong promoter to direct transcription, a ribosome binding site for translational initiation (internal ribosomal binding sequences), and a transcription/translation terminator. For E. coli, this includes a promoter such as the T7, trp, lac, or lambda promoters, a ribosome binding site, and preferably a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by well-known methods such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be cotransformed with polynucleotide sequences encoding a SARS-CoV or SARS-CoV-2 S, M, N or E binding protein or fragment thereof, or an antibody, antibody binding fragment, or conjugate that specifically binds SARS-CoV or SARS-CoV-2 S, M, N or E protein, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Viral Expression Vectors, Springer press, Muzyczka ed., 2011). One of skill in the art can readily use an expression system such as plasmids and vectors of use in producing proteins in cells including higher eukaryotic cells such as the COS, CHO, HeLa and myeloma cell lines.

When the binding protein is an antibody or antigen binding fragment, such antibodies and antigen binding fragments can be expressed as individual V_{H} and/or V_{L} chain (linked to an effector molecule or detectable marker as needed), or can be expressed as a fusion protein. Methods of expressing and purifying antibodies and antigen binding fragments are known and further described herein (see, e.g., Al-Rubeai (ed), Antibody Expression and Production, Springer Press, 2011). The nucleic acid sequences can optionally encode a leader sequence.

To create a scFv the V_{H}- and V_{L}-encoding DNA fragments can be operatively linked to another fragment encoding a flexible linker, e.g., encoding the amino acid sequence (Gly4-Ser)₃, such that the V_{H} and V_{L} sequences can be expressed as a contiguous single-chain protein, with the V_{H} and V_{L} domains joined by the flexible linker (see, e.g., Bird et al., Science 242:423-426, 1988; Huston et al., Proc. Natl. Acad. Sci. 85:5879-5883, 1988; McCafferty et al., Nature 348:552-554, 1990; Kontermann and Dubel (Ed), Antibody Engineering, Vols. 1-2, 2nd Ed., Springer Press, 2010; Harlow and Lane, Antibodies: A Laboratory Manual, 2nd, Cold Spring Harbor Laboratory, New York, 2013). Optionally, a cleavage site can be included in a linker, such as a furin cleavage site.

The nucleic acid encoding a V_{H} and/or V_{L} optionally can encode an Fc domain (immunoadhesin). The Fc domain can be an IgA, IgM or IgG Fc domain. The Fc domain can be an optimized Fc domain, as described in U.S. Published Patent Application No. 20100/093979, incorporated herein by reference. In one example, the immunoadhesin is an IgG₁ Fc.

The single chain antibody may be monovalent, if only a V_{H} and V_{L} are used, bivalent, if two V_{H} and V_{L} are used, or polyvalent, if more than two V_{H} and V_{L} are used. Bispecific or polyvalent antibodies may be generated that bind specifically to SARS-CoV S, M, N and/or E protein and/or another antigen.

Methods for expression of binding proteins, such as antibodies and antigen binding fragments, and/or refolding to an appropriate active form, from mammalian cells, and bacteria such as L coil have been described and are well-known and are applicable to the antibodies disclosed herein. See, e.g., Harlow and Lane, Antibodies: A Laboratory Manual, 2nd, Cold Spring Harbor Laboratory, New York, 2013, Simpson ed., Basic methods in Protein Purification and Analysis: A laboratory Manual, Cold Harbor Press, 2008, and Ward et al., Nature 341:544, 1989.

Also provided is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one example of the disclosure, the population of cells is a clonal population comprising host cells comprising a recombinant expression vector as described herein

Modifications can be made to a nucleic acid encoding a polypeptide described herein without diminishing its biological activity. Some modifications can be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly His) to aid in purification steps. In addition to recombinant methods, the immunoconjugates, effector moieties, and antibodies of the present disclosure can also be constructed in whole or in part using standard peptide synthesis well known in the art.

In several examples, the nucleic acid molecule encodes a precursor of the binding proteins of the present disclosure that can be processed into the SARS-CoV or SARS-CoV-2 protein or fragment thereof when expressed in an appropriate cell. For example, the nucleic acid molecule can encode a binding protein of the present disclosure including a N-terminal signal sequence for entry into the cellular secretory system that is proteolytically cleaved in the during processing of the SARS-CoV or SARS-CoV-2 protein or fragment thereof in the cell.

The polynucleotides encoding binding proteins of the present disclosure can include a recombinant DNA which is incorporated into a vector into an autonomously replicating plasmid or virus or into the genomic DNA of a prokaryote or eukaryote, or which exists as a separate molecule (such as an mRNA or a cDNA) independent of other sequences. The nucleotides can be ribonucleotides, deoxyribonucleotides, or modified forms of either nucleotide. The term includes single and double forms of DNA. In one non-limiting example, a disclosed immunogen is expressed using the pVRC8400 vector (described in Barouch et al., J. Virol, 79, 8828-8834, 2005, which is incorporated by reference herein).

Once expressed, a binding protein of the present disclosure, or an antibody, antibody binding fragment, specifically binds an epitope on a SARS-CoV or SARS-CoV-2 protein can be purified according to standard procedures in the art, including ammonium sulfate precipitation, affinity columns, column chromatography, and the like (see, generally; Simpson ed., Basic methods in Protein Purification and Analysis: A laboratory Manual, Cold Harbor Press, 2008). The SARS-CoV or SARS-CoV-2 protein or fragment thereof, or an antibody or antibody binding fragment, that specifically binds to an epitope on SARS-CoV or SARS-CoV-2 does not need to be 100% pure.

Often, functional heterologous proteins from E. coli or other bacteria are isolated from inclusion bodies and require solubilization using strong denaturants, and subsequent refolding. During the solubilization step, as is well known in the art, a reducing agent must be present to separate disulfide bonds. An exemplary buffer with a reducing agent is: 0.1 M Tris pH 8, 6 M guanidine, 2 mM EDTA, 0.3 M DIE (dithioerythritol). Reoxidation of the disulfide bonds can occur in the presence of low molecular weight thiol reagents in reduced and oxidized form, as described in Saxena et al., Biochemistry 9: 5015-5021, 1970, and especially as described by Buchner et al., supra.

In addition to recombinant methods, the binding protein, including any antibodies or antigen binding fragments can also be constructed in whole or in part using standard peptide synthesis. Solid phase synthesis of the polypeptides can be accomplished by attaching the C-terminal amino acid of the sequence to an insoluble support followed by sequential addition of the remaining amino acids in the sequence. Techniques for solid phase synthesis are described by Barany & Merrifield, The Peptides: Analysis, Synthesis, Biology. Vol. 2: Special Methods in Peptide Synthesis, Part A. pp. 3-284; Merrifield et al., J. Am. Chem. Soc. 85:2149-2156, 1963, and Stewart et al., Solid Phase Peptide Synthesis, 2nd ed., Pierce Chem. Co., Rockford, Ill., 1984. Proteins of greater length may be synthesized by condensation of the amino and carboxyl termini of shorter fragments. Methods of forming peptide bonds by activation of a carboxyl terminal end (such as by the use of the coupling reagent N,N′-dicyclohexylcarbodiimide) are well known in the art.

### Compositions and Administration

The binding proteins of Formula I or II can be included in a pharmaceutical composition (including therapeutic and prophylactic formulations), often combined together with one or more pharmaceutically acceptable vehicles and, optionally, other therapeutic ingredients (for example, antibiotics or antiviral drugs). The compositions are useful, for example, for example, for the treatment or detection of a SAILS-CoV or SARS-CoV-2 infection or induction of an immune response to SAILS-CoV or SARS-CoV-2 infection in a subject.

The compositions can be prepared in unit dosage forms for administration to a subject. The amount and timing of administration are at the discretion of the treating physician to achieve the desired purposes. The disclosed binding proteins, or a polynucleotide encoding such molecules can be formulated for systemic or local administration. In one example, the disclosed binding proteins that specifically binds to an epitope on SARS-CoV or SARS-CoV-2, or polynucleotide encoding such molecules is formulated for parenteral administration, such as intravenous administration.

The disclosed binding proteins, or polynucleotide encoding such molecules, or a composition including such molecules, as well as additional agents, can be administered to subjects in various ways, including local and systemic administration, such as, e.g., by injection subcutaneously, intravenously, intra-arterially, intranasally, intraperitoneally, intramuscularly, intradermally, or intrathecally. In an example, a therapeutic agent is administered by a single subcutaneous, intravenous, intra-arterial, intraperitoneal, intramuscular, intradermal or intrathecal injection once a day. The therapeutic agent can also be administered by direct injection at or near the site of disease.

In some aspects, the composition is administered by inhalation (e.g., by aerosol delivery), such as by use with a nebulizer such as a vibrating mesh nebulizer. In other aspects, the composition can be used with a dry power inhaler or metered dose inhaler.

A further method of administration is by osmotic pump (e.g., an Alzet pump) or mini-pump (e.g., an Alzet mini-osmotic pump), which allows for controlled, continuous and/or slow-release delivery of the therapeutic agent or pharmaceutical composition over a pre-determined period. The osmotic pump or mini-pump can be implanted subcutaneously, or near a target site.

The therapeutic agent or compositions thereof can also be administered by other modes. Determination of the most effective mode of administration of the therapeutic agent or compositions thereof is within the skill of the skilled artisan. The therapeutic agent can be administered as pharmaceutical formulations suitable for, e.g., oral (including buccal and sub-lingual), rectal, nasal, topical, pulmonary, vaginal or parenteral administration, or in a form suitable for administration by inhalation or insufflation. Depending on the intended mode of administration, the pharmaceutical formulations can be in the form of solid, semi-solid or liquid dosage forms, such as tablets, suppositories, pills, capsules, powders, liquids, suspensions, emulsions, creams, ointments, lotions, and the like.

In some aspects, the composition can be provided in unit dosage form for use to induce an immune response in a subject, for example, to prevent, inhibit, or treat SARS-CoV or SAILS-CoV-2 infection in the subject. A unit dosage form contains a suitable single preselected dosage for administration to a subject, or suitable marked or measured multiples of two or more preselected unit dosages, and/or a metering mechanism for administering the unit dose or multiples thereof. In other examples, the composition further includes an adjuvant.

A typical composition for intravenous administration of a binding protein of formula I or IT includes about 0.01 to about 30 mg/kg of per subject per day. Actual methods for preparing administrable compositions will be known or apparent to those skilled in the art and are described in more detail in such publications as Remington's Pharmaceutical Science, 19th ed., Mack Publishing Company, Easton, Pa. (1995).

To formulate the pharmaceutical compositions, the disclosed binding proteins that specifically binds to an epitope on SARS-CoV or SARS-CoV-2 protein, or polynucleotide encoding such molecules, can be combined with various pharmaceutically acceptable additives, as well as a base or vehicle for dispersion of the conjugate. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, TWEEN®80), solubility enhancing agents (for example, cyclodextrins and derivatives thereof), stabilizers (for example, serum albumin), and reducing agents (for example, glutathione) can be included.

The compositions for administration can include a solution of the disclosed the disclosed binding proteins, or polynucleotide encoding such molecules dissolved in a pharmaceutically acceptable carrier, such as an aqueous carrier. A variety of aqueous carriers can be used, for example, buffered saline and the like. The compositions may contain pharmaceutically acceptable auxiliary substances or excipients as required to approximate physiological conditions such as pH adjusting and buffering agents, toxicity adjusting agents and the like, for example, sodium acetate, sodium chloride, potassium chloride, calcium chloride, sodium lactate and the like. The concentration of the disclosed the disclosed binding proteins that specifically binds to an epitope SARS-CoV or SARS-CoV-2 protein, or polynucleotide encoding such molecules in these formulations can vary widely, and will be selected primarily based on fluid volumes, viscosities, body weight and the like in accordance with the particular mode of administration selected and the subject's needs.

The disclosed binding proteins, or polynucleotide encoding such molecules can be provided in lyophilized form and rehydrated with sterile water before administration, although they are also provided in sterile solutions of known concentration.

### Flexible Linkers

In any of the flexibly linked ACE2 decoys described herein, a flexible linker may be included. Any appropriate flexible linker may be used between each ACE2 region and the Fc region, particularly those having a length of greater than 5 nm (for a total length between the two ACE2 regions of greater about 14 nm or greater). As used herein, a flexible linker may provide a degree of movement between each ACE2 region and the Fc region. The flexible linkers may generally be composed of small, non-polar (e.g. Gly) or polar (e.g. Ser or Thr) amino acids. The small size of these amino acids may provide flexibility, and may allow for mobility of the connecting region. The incorporation of Ser or Thr can maintain the stability of the linker in aqueous solutions by forming hydrogen bonds with the water molecules, and may reduce the unfavorable interaction between the linker and the protein moieties.

The flexible linkers may have sequences consisting primarily of stretches of Gly and Ser residues (“GS” linker). One example of a flexible linker has the sequence of (Gly-Gly-Gly-Gly-Ser)ₙ. By adjusting the copy number “n”, the length of this GS linker can be adjusted to achieve appropriate separation of the functional regions e.g., ACE2 regions or other binding regions). Other flexible linkers may be rich in small or polar amino acids such as Gly and Ser, but can contain additional amino acids such as Thr and Ala to maintain flexibility, as well as polar amino acids such as Lys and Glu to improve solubility. Other types of flexible linkers, include KESGSVSSEQLAQFRSLD and EGKSSGSGSESKST. These linkers may be repeated (KESGSVSSEQLAQFRSLD)ₙ or (EGKSSGSGSESKST)ₙ. Another flexible linker is GSAGSAAGSGEF, or (GSAGSAAGSGEF)ₙ. In any of these linkers the length of the linker may be adjusted by selecting the number of repeats, n (e.g., n is 1, 2, 3, 4, 5, 6, 7, 8, 9, 10, 11, 12, 13, 14, 15, 16, 17, 18, 19, 20, 21, 22, 23, 24, 25, etc.). The length of each linker, e.g., between the ACE2 region and the Fc region, may be selected so that the total separation of the ACE2 domains (or in some examples, the ACE2 domain and another COV

### Mucotrapping

As used herein, the term “trapping potency” refers to the ability of a binding protein (e.g., the binding proteins described herein) that specially binds to a target pathogen to inhibit movement of the pathogen through mucus. Trapping potency can be measured by methods known in the art and as disclosed herein. Trapping potency can be quantitated, e.g., as the amount of binding protein (e.g., concentration of binding protein in mucus) needed to reduce the mobility of at least 50% (e.g., at least 55%, at least 60%, at least 65%, at least 70%, at least 75%, etc.) of the pathogen within the mucus gel to at least one-half (e.g., one-quarter, one-tenth, etc.) of its native mobility in solution (e.g., saline) and/or in mucus. Mobility in mucus can be measured using techniques well known in the art and described herein. Alternatively, trapping potency can be quantitated as the reduction in percentage of pathogens that penetrate mucus.

The term “enhances trapping potency” refers to enhancement compared to the protein (e.g., to the Fc domain in the flexibly linked ACE2 decoys described herein). Further, any of binding proteins described herein may be selected or further configured to enhance mucin-crosslinking by including a glycosylation pattern comprising the biantennary core glycan structure Manα1-6(Manα1-3)Manβ1-4GlcNAcβ1-4GlcNAcβ1 with terminal N-acetylglucosamine on each branch. This glycosylation pattern may be on the Fc region of the protein (e.g., of the flexibly linked ACE2 decoy). Alternatively or additionally, a composition of the constructs described herein may be selected or configured such that at least x % of the constructs (e.g., the dimerized flexibly linked ACE2 decoy binding proteins) has a glycosylation pattern comprising the biantennary, core glycan structure Manα1-6(Manα1-3)Manβ1-4GlcNAcβ1-4GlcNAcβ1 with terminal N-acetylglucosamine on each branch, where x % is 20%, 25%, 30%, 35%, 40%, 45%, 50%, 55%, 60%, 65%, 70%, 75%, 80%, 85%, 90%, 95%, or substantially all). A composition in which, for example, greater than 20% (greater than 25%, greater than 30%, greater than 35%, greater than 40%, greater than 45%, greater than 50%, etc.) of the constructs described herein include an oligosaccharide that provides increased mucin crosslinking (e.g., G0), may be particularly beneficial for muco-trapping of a target once bound to the target (e.g., a SARS-like CoV).

The binding proteins, including the flexibly linked ACE2 decoys, compositions, and methods described herein may include methods for inhibiting and/or treating infection by a SARS-like CoV (and in particular SARS-CoV-2), and/or eliminating pathogen from a mucosal surface. In particular, the presently-disclosed subject matter relates to constructs and compositions of these that are capable of facilitating aggregation and/or enchained growth of pathogens (e.g., SARS-CoV), and/or trapping the pathogens in mucus, thereby inhibiting transport of pathogens across or through mucus secretions, which may lead to the destruction and/or natural elimination of these pathogens.

The binding protein constructs (including the flexibly linked ACE2 decoys) described herein may generally diffuse rapidly through mucus, slowed only slightly by weak, transient adhesive interactions with mucins within the mucus. This rapid diffusion allows the constructs to accumulate pathogen. When a plurality of constructs have coupled to the pathogen, the adhesive interactions between the plurality constructs and the mucus may become sufficient to trap the bound pathogen in the mucus, thereby preventing or reducing infection. Pathogens trapped in mucus cannot reach target cells in the body, and will instead be shed and/or inactivated by spontaneous thermal degradation as well as additional protective factors in mucus, such as defensins. As disclosed herein, this pathogen agglutination and/or trapping activity provides for protection without neutralization, and can effectively inhibit infection even at relatively low doses. The low-affinity interactions that the constructs described herein may form with mucins may also be influenced by glycosylation.

Thus, the constructs described herein may include an oligosaccharide at a glycosylation site (in particular, on the Fc domain), the oligosaccharide comprising or consisting of (or in some examples, consisting essentially of), a pattern correlating with (providing) enhanced trapping potency of the binding protein in mucus. The binding protein specifically binds the target (e.g., SARS-like CoV target, such as SARS-CoV-2). The glycosylation pattern/oligosaccharide component of the binding protein (e.g., the flexibly linked ACE2 decoy protein) may maximize trapping potency of the binding protein once the binding protein forms a complex with one or more targets (e.g., pathogen, such as SAILS-CoV′-2), without unduly hindering the ability of the unbound constructs to diffuse readily through mucus to rapidly bind a target. In certain examples, the constructs described herein exhibit a mobility in mucus that is reduced no more than about 50%, e.g., no more than about 40%, 30%, 20%, 10%, or 5%, relative to its native mobility in solution (e.g., mucus, saline or water) and effectively traps a target pathogen in mucus once complexed with one or more targets (e.g., at least 50% of target slowed by at least on half). In some examples, the constructs described herein reduces the mobility of at least 50% of the target, e.g., at least 50%, 60%, 70%, 80%, or 90% or more of the target, by at least 50% (e.g., 60%, 70%, 75%, 80%, 85%, 90%, 95%, 96%, 97%, 98%, or 99%, etc.) or more. In other examples, the constructs described herein reduces the percentage of target (e.g., pathogens) that can penetrate mucus by at least 10%, e.g., at least 20%, 30%, 40%, 50%, 60%, 70%, 80%, 90%, or more. For example, the constructs described herein may have a sufficient binding rate to an epitope of the target to trap the target pathogen in mucus within one hour (e.g., within 30 minutes or 15 minutes) at a construct concentration in the mucus of less than 10 mg/ml (e.g., less than 5 mg/ml, less than 1 mg/ml, less than 0.1 mg/rill, less than 50 μg/ml, less than 30, less than 20, less than 10, less than 5, less than 2.5, less than 1, less than 0.5, less than 0.1 μg/ml, etc.).

In some examples, the constructs described herein may include an oligosaccharide component that is bound to an N-linked glycosylation site in an Fc region of the constructs. The N-linked glycosylation site can be an asparagine residue on the Fc region, for example, the Asn 297 asparagine residue. The amino acid numbering is with respect to the standard amino acid structure of a human/humanized IgG molecule. As mentioned, Fc regions from IgM, IgD, IgG, IgA and IgE, or modified variants thereof, may be used.

The N-glycan structure may be G0/G0F form, or a pure GnGn form (e.g., with terminal N-acetylglucosamine on each branch without terminal galactose or sialic acid). In some examples, the oligosaccharide component, i.e., the glycan, attached to the construct comprises, consists essentially of, or consists of a core structure without any fucose residue. In some examples, the oligosaccharide component comprises fucose on a side chain. In other examples, the glycan does not contain any galactose residues. In some examples the glycan does not include galactose.

The constructs described herein may include a mixture of constructs having different oligosaccharide components. In some examples, the mixture comprises at least about 30% constructs having the G0/G0F core glycan structure (e.g., with or without the fucose residue), e.g., at least about 40%, 50%, 60%, 70%, 80%, 90% or more.

In some examples, the constructs described herein are generated in a human cell line, e.g., a 293 cell line, e.g., a 293T cell line, other mammalian cell lines (e.g. CHO), in plants (e.g. Nicotiana), or in other microorganisms (e.g. Trichoderma).

The constructs described herein may be useful for binding target to trap the target in mucus to inhibit infection by the target. The constructs described herein can be used to treat, prevent or reduce infection by any virus that binds to ACE2, such as coronaviruses (e.g., SARS-CoV-2) which may infect a subject through a mucus membrane.

The terms virus, pathogen and viral pathogen may be used interchangeably herein, and further refer to any virus that binds to ACE2, such as coronaviruses (e.g., SARS-CoV-2).

### Compositions

As would be recognized by one skilled in the art, the constructs described herein can also be formed into suitable compositions, e.g., pharmaceutical compositions for administration to a subject in order to treat or prevent an infection caused by a target pathogen (e.g., a virus that binds to ACE2, such as coronaviruses, such as SARS-CoV-2) or a disease or disorder caused by infection by a target pathogen. A composition may comprise, consist essentially of, or consist of a construct as described herein in a prophylactically or therapeutically effective amount and a pharmaceutically-acceptable carrier.

Pharmaceutical compositions containing the constructs described herein can be formulated in combination with any suitable pharmaceutical vehicle, excipient or carrier that would commonly be used in this art, including such conventional materials for this purpose, e.g., saline, dextrose, water, glycerol, ethanol, and combinations thereof. As one skilled in this art would recognize, the particular vehicle, excipient or carrier used will vary depending on the subject and the subject's condition, and a variety of modes of administration would be suitable for the compositions described herein. Suitable methods of administration of any pharmaceutical composition disclosed in this application include, but are not limited to, topical, oral, intranasal, buccal, inhalation, anal, and vaginal administration, wherein such administration achieves delivery of the binding protein to a mucus membrane of interest.

The composition can be any type of composition suitable for delivering a construct described herein to a mucosal surface and can be in various forms known in the art, including solid, semisolid, or liquid form or in lotion form, either oil-in-water or water-in-oil emulsions, in aqueous gel compositions. Compositions include, without limitation, gel, paste, suppository, douche, ovule, foam, film, spray, ointment, pessary, capsule, tablet, jelly, cream, milk, dispersion, liposomes, powder/talc or other solid, suspension, solution, emulsion, microemulsion, nanoemulsion, liquid, aerosol, microcapsules, time-release capsules, controlled release formulation, sustained release formulation or bioadhesive gel (e.g., a mucoadhesive thermogelling composition) or in other forms embedded in a matrix for the slow or controlled release of the composition to the surface onto which it has been applied or in contact.

If topical administration is desired, the composition may be formulated as needed in a suitable form, e.g., an ointment, cream, gel, lotion, drops (such as eye drops and ear drops), or solution (such as mouthwash). The composition may contain conventional additives, such as preservatives, solvents to promote penetration, and emollients. Topical formulations may also contain conventional carriers such as cream or ointment bases, ethanol, or oleyl alcohol. Other formulations for administration, including intranasal administration, etc., are contemplated for use in connection with the presently-disclosed subject matter. All formulations, devices, and methods known to one of skill in the art which are appropriate for delivering the constructs described herein or a composition containing the constructs described herein to one or more mucus membranes of a subject can be used in connection with the presently-disclosed subject matter.

Any of the compositions described herein may include mixtures of the constructs described herein.

The compositions used in the methods described herein may include other agents that do not negatively impact or otherwise affect the inhibitory effectiveness of the components of the composition, including antibodies and antiviral agents. For example, solid, liquid or a mixture of solid and liquid pharmaceutically acceptable carriers, diluents, vehicles, or excipients may be employed in the pharmaceutical compositions. Suitable physiologically acceptable, substantially inert carriers include water, a polyethylene glycol, mineral oil or petrolatum, propylene glycol, hydroxyethylcellulose, carboxymethyl cellulose, cellulosic derivatives, polycarboxylic acids, linked polyacrylic acids, such as carbopols; and other polymers such as poly(lysine), poly(glutamic acid), poly(maleic acid), polylactic acid), thermal polyaspartate, and aliphatic-aromatic resin; glycerin, starch, lactose, calcium sulphate dihydrate, terra alba, sucrose, talc, gelatin, pectin, acacia, magnesium stearate, stearic acid, syrup, peanut oil, olive oil, saline solution, and the like.

The pharmaceutical compositions described herein useful in the methods of the present invention may further include diluents, fillers, binding agents, colorants, stabilizers, perfumes, gelling agents, antioxidants, moisturizing agents, preservatives, acids, and other elements known to those skilled in the art. For example, suitable preservatives are well known in the art, and include, for example, methyl paraben, propyl paraben, butyl paraben, benzoic acid and benzyl alcohol.

For injection, the carrier may typically be a liquid, such as sterile pyrogen-free water, pyrogen-free phosphate-buffered saline solution, bacteriostatic water, or Cremophor EL® (BASF, Parsippany, N.J.). For other methods of administration, the carrier can be either solid or liquid.

For oral administration, the constructs described herein can be administered in solid dosage forms, such as capsules, tablets, and powders, or in liquid dosage forms, such as elixirs, syrups, and suspensions. Compositions can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Compositions suitable for buccal (sub-lingual) administration include tablets or lozenges comprising the binding protein in a flavored base, usually sucrose and acacia or tragacanth; and pastilles comprising the binding protein in an inert base such as gelatin and glycerin or sucrose and acacia. The composition can comprise an orally dissolvable or degradable composition. Alternately, the composition can comprise a powder or an aerosolized or atomized solution or suspension comprising the binding protein. Such powdered, aerosolized, or atomized compositions, when dispersed, preferably have an average particle or droplet size in the range from about 0.1 to about 200 nanometers.

Compositions of the constructs described herein that are suitable for parenteral administration comprise sterile aqueous and non-aqueous injection solutions of the constructs described herein, which preparations are preferably isotonic with the blood of the intended recipient. These preparations can contain antioxidants, buffers, bacteriostats and solutes which render the composition isotonic with the blood of the intended recipient. Aqueous and non-aqueous sterile suspensions can include suspending agents and thickening agents. The compositions can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect, there is provided an injectable, stable, sterile composition comprising a construct described herein, in a unit dosage form in a sealed container. The constructs described herein may be provided in the form of a lyophilizate which is capable of being reconstituted with a suitable pharmaceutically acceptable carrier to form a liquid composition suitable for injection thereof into a subject.

Compositions suitable for rectal administration may be presented as unit dose suppositories. These can be prepared by admixing the constructs described herein with one or more conventional solid carriers, for example, cocoa butter, and then shaping the resulting mixture.

In particular, the constructs described herein can alternatively be formulated for nasal administration or otherwise administered to the lungs of a subject by any suitable means, e.g., administered by an aerosol suspension of respirable particles comprising the constructs described herein, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Harwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the constructs described herein can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the constructs described herein can likewise be produced with any solid particulate medicament aerosol generator, by techniques known in the pharmaceutical art.

Alternatively, one can administer the constructs described herein in a local rather than systemic manner, for example, in a depot or sustained-release formulation.

The constructs described herein may be coated or impregnated on a device (or a composition including the constructs described herein may be coated or impregnated). The device can be for delivery of the constructs described herein and compositions of the synthetic binding agent to a mucus membrane (including the lungs, nose, mouth, etc.).

As noted, constructs described herein is capable of diffusing through mucus when it is unbound, to allow the constructs to bind a target (e.g., pathogen) at a desirable rate. It is also desirable that, when constructs described herein is bound to the target, the cumulative effect of the binding protein-mucin interactions effectively traps the pathogen in the mucus and/or agglutinates the target.

In some examples, the pharmaceutical composition can further include an additional active agent, e.g., a prophylactic or therapeutic agent. Suitable antiviral agents include, for example, virus-inactivating agents such as nonionic, anionic and cationic surfactants, and C31 G (amine oxide and alkyl betaine), polybiguanides, docosanol, acylcarnitine analogs, octyl glycerol, and antimicrobial peptides such as magainins, gramicidins, protegrins, and retrocyclins. Mild surfactants, e.g., sorbitan monolaurate, may advantageously be used as antiviral agents in the compositions described herein. Other antiviral agents that may advantageously be utilized in the compositions described herein include nucleotide or nucleoside analogs, such as tenofovir, acyclovir, amantadine, didanosine, foscarnet, ganciclovir, ribavirin, vidarabine, zalcitabine, and zidovudine. Further antiviral agents that may be used include non-nucleoside reverse transcriptase inhibitors, such as UC-781 (thiocarboxanilide), pyridinones, nevaripine, delavirdine, calanolide A, capravirine and efavirenz. From these reverse transcriptase inhibitors, agents and their analogs that have shown poor oral bioavailability are especially suitable for administration to mucosal tissue.

The presently-disclosed subject matter further includes a kit including the constructs described herein or a composition comprising the constructs as described herein; and optionally a device for administering the constructs or composition.

### Examples

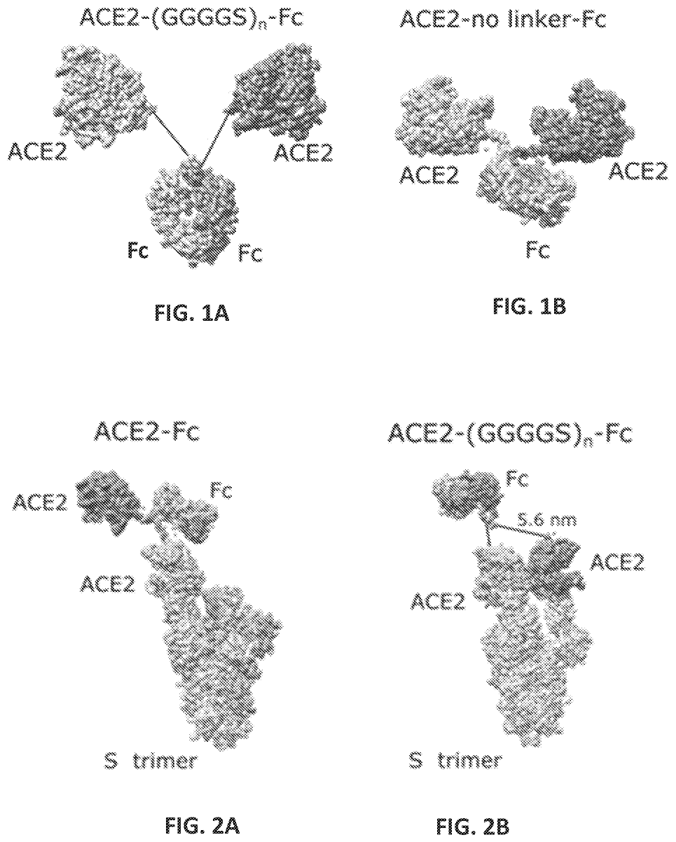

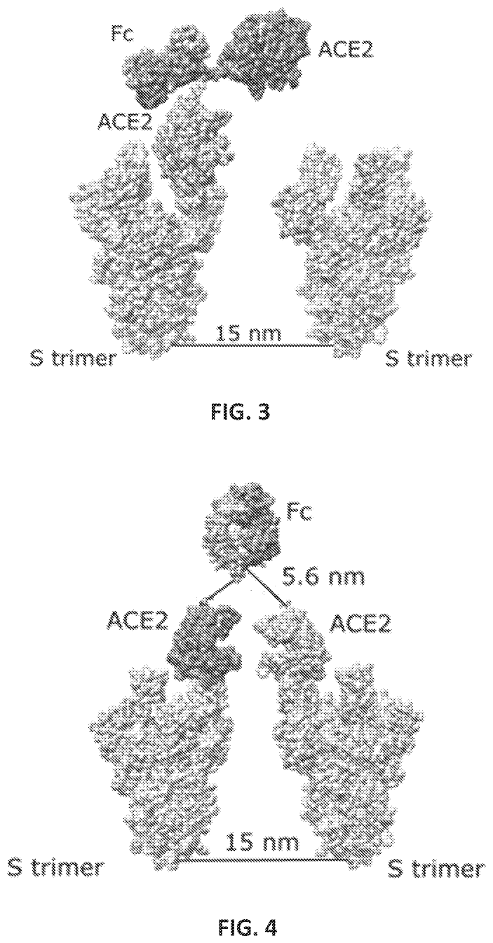

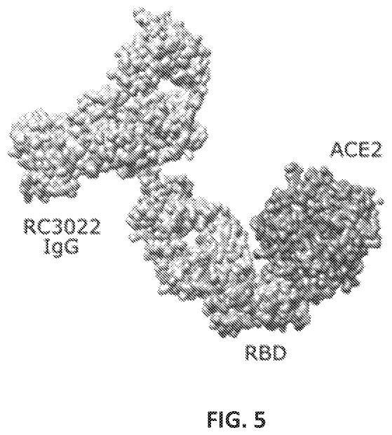

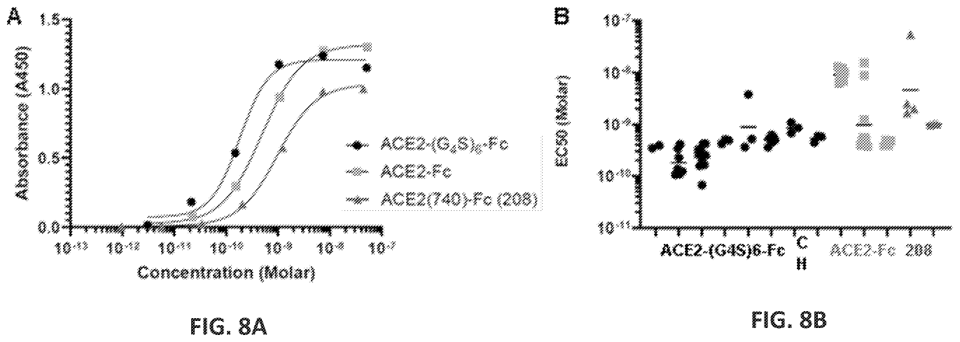

The family of viruses that bind to ACE2 includes SARS-CoV-2, SARS-CoV-1 and NL63-CoV. These viruses gain entry into cells when the receptor binding domain (RBD) of their spike protein (S) binds to angiotensin-converting enzyme 2 (ACE2) on the target cell's surface. This ACE2-tropism may be used to develop ACE2-Fc decoys that can neutralize the virus. One strategy has been to link the entire ACE2 molecule (residues 18-740, which includes the self-dimerizing collectrin domain) to human IgG1-Fc, or linking simply the extracellular segment of ACE2 without the C-terminal collectrin domain (residues 18-614) to human IgG1-Fc. See, e.g., FIGS. 6A and 6B. However, S-proteins only bind ACE2 with modest affinity, thus the corresponding neutralizing potency of such ACE2-decoys based on wildtype ACE2 is limited; typical binding affinity (EC50s) and neutralization potencies (IC50s) range from hundreds of rig/mL to tens of ug/mL range. Such potencies are at minimum roughly 1 to 2 log worse than those of monoclonal antibodies that have received EUA or are under active clinical development. See, e.g., FIGS. 1B and 2A, showing ACE-Fc dimers.

To overcome the limited affinity of wildtype ACE2 to S proteins, higher affinity ACE2 variants have been engineered by random mutagenesis and selection using yeast surface display (see, e.g., Table 1, FIGS. 16A-16B). The directed evolution strategy leaves open the possibility of an escape virus that binds wildtype ACE2 but not the mutated ACE2. Thus, another approach that can improve the binding affinity yet utilizes naturally occurring ACE2 may be beneficial. Cryo-electron microscopy of SARS-CoV-1 shows that ˜50-100 S proteins are present on the virus surface, with an average spacing of ˜15 nm. The trimeric form of the 5-protein spike also results in a large distance between any 2 of the 3 S proteins on an individual spike. In both cases, the distance limits the two Fab domains on an antibody from binding to two distinct S-proteins at the same time. Given the high sequence homology between viruses that bind to ACE2 (e.g., SARS-CoV-1 and SARS-CoV-2), the presentation of S proteins on such viruses is likely similar. Thus, the methods and compositions described herein may improve the potency of ACE2 decoys by tuning the presentation of the two ACE2 domains to maximizes the likelihood of achieving bivalent binding on the surface of the virus.

Cryo-EM analysis suggest most spike proteins have either 1 or 2 RBDs in the “up” conformation, and exceedingly few have all 3 RBDs of the same spike assuming the “up” conformation. Native ACE2 is a homo-dimer, with the collectrin domain serving as the major dimerization domain. The work performed herein suggests that this geometry may preclude the molecule from achieving optimal intra-spike binding. To overcome this shortcoming, an Fc domain (such as, but not limited to the VH-CH1 domain of a standard IgG1 Fab) may be combined with the extracellular domain of ACE2, excluding the collectrin domain (residues 18-614), and further include an extended (e.g., 22 or more, 23 or more 24 or more, 25 or more, 26 or more, 27 or more, 28 or more. 29 or more, 30 or more, 31 or more, 32 or more, 33 or more, 34 or more, 35 or more, etc.) amino acid, flexible linker between the ACE2 fragment and the Fc domain (the Fc constant heavy chain domain, CH2) designed to increase the reach of the molecule and consequently greater binding affinity. See, E.g., FIG. 6C (and FIGS. 1A and 2B).

As described herein, flexibly linked flexibly linked ACE2 decoy constructs (e.g., ACE2-(G₄S)₆-Fc, as shown in FIG. 6C) binds different variants of SARS-CoV-2 S proteins, neutralize SARS-CoV-2 pseudovirus with picomolar potencies, effectively traps SARS-CoV-2 virus like particles in human airway mucus, can be stably nebulized, and effectively reduces SARS-CoV-2 infections in hamsters.

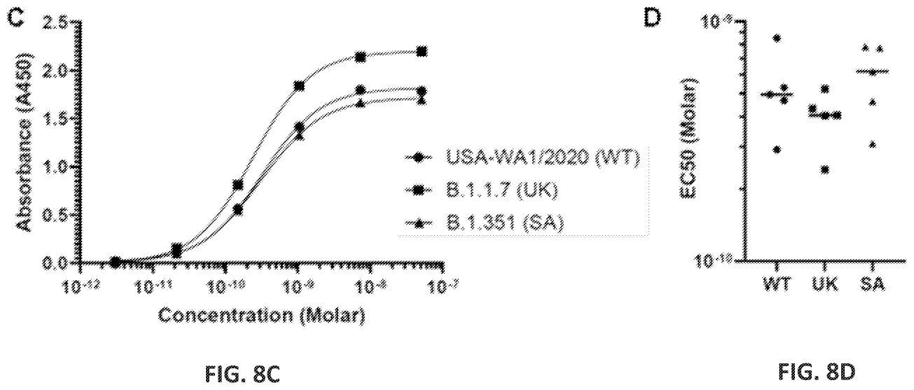

To determine the distance between ACE molecules bound to the same S protein, the reported spike protein structure from 7A98 was generated in the “three up” RBDs conformation. This model was used to determine if ACE2-Fc FIGS. 1B and 2A) could bind to two RBDs on the same S protein trimer. See, e.g., FIG. 7A. As shown in FIG. 7A, when one of the two ACE2 domain on ACE2-Fc engages any one of the three RBD, the remaining ACE2 domain becomes oriented upwards away from the S protein, due to the lack of flexibility and length in the hinge of IgG1 connecting ACE2 to Fc. Thus, it is not likely that the ACE2-Fc can efficiently bind bivalently to either two RBDs on the same Spike protein or RBDs between two different Spike proteins on the virus. The same limitation holds for the conventional ACE2-Fc encompassing the collectrin domain, since collectrin domain dimerization directly limits the reach of the adjacent ACE2 fragments, as shown in FIG. 6A.

The estimated distances between RBDs on the same spike protein ranged from 60 to 100 Å when these three RBDs are in the “three-up” conformation. To bridge the distance and add flexibility to the molecule, a flexible linker (such as, but not limited to (GGGGS)₆ flexible linkers) were added, with a length of ˜10 nm, between extracellular ACE2 fragment and the Fc region (e.g., the IgG1-Fc), as shown for one example of a flexibly linked ACE2 decoy construct (e.g., in FIG. 6C, ACE2-(G₄S)₆-Fc). Since the flexible linker is present on each of the two heavy chains, the two ACE2 fragments on the flexibly linked ACE2 decoy (e.g., ACE2-(G₄S)₆-Fc) can theoretically span distances nearly twice that length, i.e. —20 nm. Modeling suggests that flexibly linked ACE2 decoys in which the flexible linker is sufficiently long (e.g., ACE2-(G₄S)₆-Fc is but one example) have the necessary flexibility and reach to bind bivalently when any two RBDs are oriented in the “up” conformation, as shown in FIG. 7B.

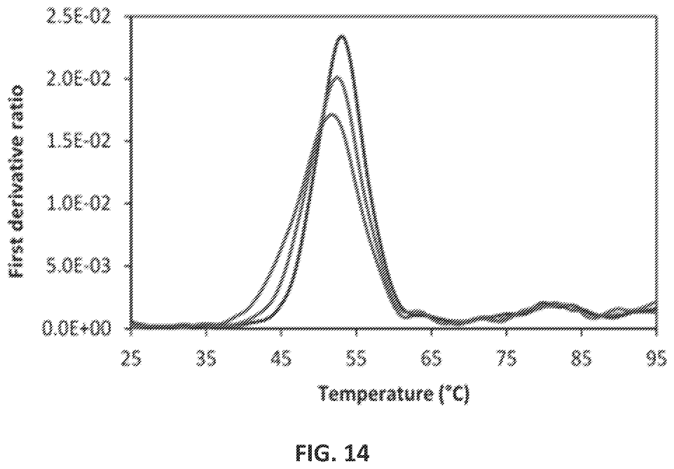

This relationship was examined for both ACE2-Fc and ACE2-(G₄S)₆-Fc in mammalian culture, and both molecules were purified using standard Protein A chromatography. The molecules were examined in Native-PAGE (see, FIG. 7C); the single bands for both confirm that they exist as monomers, although they ran at a molecular weight ˜350 kDa. Their molecular weight was examined using Size Exclusion Chromatography/Multi-Angle Light Scattering (SEC/MALS). ACE2-Fc and ACE2-(G₄S)₆-Fc possessed MW of ˜208 kDa and ˜212 kDa, respectively, in good agreement with the theoretical MW, as shown in FIG. 7D. Both molecules are predominantly found in the monomeric form: ACE2-Fc and ACE2-(G₄S)₆-Fc were ˜85% and ˜91% monomer after simple Protein A purification, respectively, with the remainder fraction corresponding to oligomers of the proteins and aggregates. Appreciably greater yields were consistently obtained with ACE2-(G₄S)₆-Fc production, with an average amount of ˜86 mg per 500 mL of culture, which is more than double the typical yield achieved with producing ACE2-Fc under identical conditions, which yields ˜36 mg of protein per 500 mL of culture. For example, see FIG. 13.

### Methods

The ACE2 decoys (including the flexibly linked ACE2 decoys) described herein were cloned from plasmids containing ACE2 without a CD domain fused to monomeric Fc domain (pAce2-mFc). Double stranded DNA strings, Gblocks®, containing (GGGGS)₆-Fc fusion were purchased from 1D′T DNA. To generate the plasmid for ACE2-(G₄S)₆-Fc (pAce2-LdFc), pAce2-mFc was digested with BamH and XhoI, and (GGGGS)₆—Fc was inserted by Gibson assembly. The reaction was transformed to chemically competent TOP10® (Thermo Fisher) and plated in LB+carbenicillin plates. Sanger sequencing was used to confirm assembly. To generate the plasmid for ACE2-Fc (pAce2-dFc), Fc was amplified from (GGGGS)₆-Fc Gblock® using primers pF1 and pR1 using high-fidelity Phusion polymerase. The PCR product was then cloned into pAce2-mFc digested with BamH and XhoI by Gibson assembly, as previously described.

Cloning of SARS-CoV-2 wild type and mutant S proteins for soluble expression was done from plasmid nCov-2.sol, which encodes SARS-CoV-2 wild type S protein with 2P mutations, a mutated furin site, a C-terminal foldon and hexa-histidine tag. This was used for soluble expression of S protein. To generate S proteins encoding the mutations in the South African strain of SARS-CoV-2 SA in nCov2.sol, the plasmid was digested with AgeI and NheI. PCR primers Pf2,Pr2. Pf3,pR3 were designed to amplify 2 fragments from S protein with mutations K417N, E484K, and N501Y. These two fragments were cloned into digested nCoV2.sol by Gibson assembly to generate a full-length S protein with SA mutations. Proper assembly of the protein was confirmed by Sanger sequencing (Genewiz). Hexapro mutations intended to stabilize the soluble protein were inserted into wild type and SA-nCov2.sol by amplifying nCov2.sol with primers Pf5 and Pry, which amplify the vector and S protein 1-816 and 943-1208, and inserting a DNA fragment encoding S protein residues 817-942 with hexa-pro mutations by Gibson assembly. The resulting vectors are henceforth referred to as WT-hexapro-nCov2.sol and SA-hexapro-nCov2.sol. The fragment with hexa-pro mutations was amplified from plasmid UK-hexapro-nCoV2.xdna. This plasmid encodes for the S protein of the UK strain with hexa-pro mutations and was purchased from Twist Bioscience.

Plasmids needed for the generation of SARS-CoV-2 pseudotyped infectious lentivirus were generated as follows. The plasmid pUC57-2019-nCoV-S containing human codon optimized Spike DNA was purchased from Genscript Molecular Cloud. This DNA was amplified using primers (P6, P6) to generate a C-terminal truncation, and cloned into the mammalian expression vector pAH to generate pAH-S-CoV-2-ΔCt. To generate pAH-S-Cov2-ΔCt with SA mutations, pAH-SA-CoV-2-ΔCt.vlp, one fragment of SA S protein was amplified from SA-nCov1.sol using primers Pf7 and Pr7. Another fragment of WT S protein was amplified with Pf8 and Pr8 from WT pAH-S-CoV-2-ΔCt. The fragments were then cloned into pAH cloning vector digested with KpnI and XhoI by Gibson assembly. Lentivirus was made using a third generation packaging system using 4 plasmids pMDLg/pRRE (Addgene)+pRSV-Rev (Addgene)+pAH-S-CoV-2-ΔCt and a transfer plasmid 7 EGFP) containing EGFPgene which was used to track infection.

Plasmids needed for generation of non-replicating SARS-Cov-2 wild type and SA VLPS were generated as follows. A Gblock® encoding the C-terminal domain of SARS-CoV-1 was purchased from IDT DNA. WT-hexapro-nCov2.sol, SA-hexapro-nCov2.sol, and UK-hexapro-nCov2.sol were digested with BamHI and XhoI to remove the foldon domain and his tag, and then the Gblock® containing the Cterm of SARS-CoV-1 was introduced by Gibson assembly.

Endotoxin free pAce2-dFc and pAce2-LdFc for transfections were purified using NucleoBond Xtra Midi Plus EF kit (Macherey-Nagel). ACE2-Fc and ACE2-(G4S)6-Fc were produced in Expi293T™ cells by transient transfection using ExpiFectamine™ Transfection Kit (Thermo Fisher). 500 mL cultures were used, and cells were harvested before viability dropped below ˜75%. Cell culture supernatants were concentrated using tangential flow (Sartorius Vivaflow 50 crossflow cassette system with 100,000 MWCO cassette with Polyethersulfone membrane) for purification by protein A chromatography. Three 5 mL HiTrap Protein A columns (Cytivia) were connected in tandem to a NGC Quest 10 FPLC (BioRad). The columns were equilibrated with 5 column volumes (CV) of 10 mM sodium phosphate buffer, pH 7.0. Protein was loaded into the column at 0.5 mL/min, followed by a 10 CV wash step with phosphate buffer, and subsequently eluted by a 5 CV isotonic elution step with 100% 0.2M glycine buffer pH 2.0. 3 mL fractions were collected in tubes filled with 300 uL of 1M Tris buffer with 0.2% polysorbate 80, pH 8. Purity of each fraction was assessed by SDS-PAGE, and the fractions with no extra bands were combined and buffer exchanged into 20 mM His, mg/mL sucrose, 0.2% polysorbate 80, 130 mM NaCl, pH 6.2 (standard buffer) using Spin-X® UF 50k MWCO PES spin columns (Corning). Following buffer exchange, the proteins were filtered with a 0.22 μm filter and flash frozen in liquid nitrogen before been stored at −80° C.

Endotoxin free ncov2.sol, WT-hexapro-nCov2.sol, SA-hexapro-nCov2.sol, and UK-hexapro-nCov2.sol were purified using NucleoBond Xtra Midi Plus EF kit. The plasmids were transfected into Expi293T™ using ExpiFectamine™ Transfection Kit. 500 mL cultures were used, and cells were harvested before viability dropped below ˜45%. Cell culture supernatants were concentrated ten-fold by tangential flow (Sartorius Vivaflow 50 crossflow cassette system with 100,000 MWCO cassette with Polyethersulfone membrane). The concentrated supernatant was incubated with 1 mL of Ni-Nta agarose resin (Qiagen) overnight before being recovered with a gravity-flow column (Bio-Rad). The resin was then washed with several column volumes of PBS with 20 mM imidazole, followed by elution with PBS with 500 mM imidazole. The proteins were then buffer exchanged into PBS or 20 mM tris with 120 mM sucrose and 20 mM sodium chloride pH 7 using Spin-X® UF 50k MWCO PES spin columns. Following buffer exchange, the proteins in tris-sucrose buffer were flash frozen in liquid nitrogen before been stored at −80° C.

Fluorescent VIPs were made by cotransfection of pGAG-mcherry plasmid (kind gift from Gummuluru lab) and Cov2 S protein plasmid in a 1:1 ratio. Non-replicating lentivirus pseudotyped with SARS-CoV-2 UK spike protein were created using the following plasmids, in a 1:1:1:2 ratio: pMDLg/pRRE, pRSV-REV, SARS-CoV-2 UK Spike, and pLL7 GFP. Non-replicating lentivirus pseudotyped with SARS-CoV-2 South African spike were created using the same plasmids/ratio above with SARS Cov2 UK spike replaced with SARS Cov2 South African spike. All plasmids were purified using NucleoBond Xtra Midi Plus EF kit. The plasmids were transfected into LVMaxx using the LVMaxx Transfection kit. Each VIP was made in 60 mL cultures, and harvested after 48 hours. The VLPs were purified using 25% Sucrose (in 25 mM Hepes/130 mM NaCl) cushion spin protocol, 3 mL of 25% sucrose solution was add to each Beckman Coulter ultracentrifuge tube, which then had 7 mL of cell culture supernatant gently, layered on top. The tubes were then spun at 36,000 rpm for 2.5 hours at 4° C. The sucrose/supernatant was then aspirated off, and 20 uL of 10% Sucrose solution was placed on top of the VLP pellet. After 24 hours at 4° C., the VLPs were then aliquoted and stored at −80° C.

The 3D models described herein were generated using UCSF Chimera 1.14 to generate all the protein models, and UCSF Chimera X 1.1 was used to render the model for publication. ACE2-Fc and ACE2-(G4S)6-Fc were constructed using models 6M17 for ACE2, 1HZH for human IgG, and 1EIB for GGGGS linker. ACE2-Fc and ACE2-(G4S)6-Fc bound to S protein were generated by matching the ACE2 of ACE2-Fc and ACE2-(G4S)6-Fc with the RBD-bound ACE2 in the “all-up” S protein model 7A98. The predicted 3D model of ACE2-Fc with collectrin domain was modified from.

SEC-MALS measurements of purified proteins and native PAGE were performed using solutions containing 1.0 mg/mL ACE2-(G4S)6-Fc or Ace2-Fc were prepared in standard buffer. 100 uL of these solutions were then loaded into a Superdex 200 Increase 10/300 GL (Cytivia) mounted on a NGC Quest 10 FPLC (BioRad). The column was pre-equilibrated with PBS, and the whole run was performed at 0.5 mL/min. The molecular weight of proteins eluting from the column was determined using a Mini Dawn multi-angle light scattering detector and its companion software Astra8 (Wyatt), assuming an extension coefficient of 1.92. Molecular weight was calculated for two independent batches of ACE2-(G4S)6-Fc and 2 batches of Ace2-Fc. For native PAGE, 5 μg of protein were loaded onto 3-12% Bis-Tris gels (Invitrogen), and the gels were ran as described by the manufacturer's protocol.

For scanning differential fluorimetry, the melting temperature of ACE2-LFC was determined by nanoDSF using a Promethius NT.48 (Nanotemper Technologies). Samples were heat up from 25° C. to 95° C. at a rate of 1° C./min. Samples were measured in triplicate. Reported data is the average of 3 independent repeats.

ELISA binding assays were performed using 96 well half-area plates (Fisher Scientific, Costar 3690) coated with 0.5 ug/mL of S protein and incubated overnight at 4° C. ELISA plates were blocked the following day with 5% (w/v) milk (LabScientific MSPP-M0841) with Tween 20 (Fisher Scientific BP337-100) at a 1:2000 dilution at room temperature for one hour. Samples were diluted in 1% (w/v) milk with Tween 20 at a 1:10,000 dilution and plated once the blocking had commenced and the 5% milk had been discarded. Samples were incubated at room temperature for 1 hr, and the solution was discarded after the 1 hr incubation. Plates were then washed with PBS containing Tween 20 at 1:2000 dilution four times. A peroxidase-conjugated goat anti-human IgG Fc antibody (Rockland 709-1317) was diluted in 1% milk with Tween 20 at a 1:5000 dilution, plated, and incubated at room temperature for 1 hr. The solution was then discarded and washed with PBS with Tween 20 two times, followed by washing with just PBS two more times. Plates were developed with TMB solution (ThermoFisher 34029), and development was stopped by adding 2N HCl (Sigma-Aldrich 320331). The absorbance at 450 nm and 595 nm was then measured with a microplate photodetector (Fisher Scientific, accuSkan FC).

For the neutralization assays described herein, a series of ten 4-fold serial dilutions were made of ACE2-Linker-Fc or ACE2-Fc or IgG starting at 20 ug/ml in OptiMEM. 10 ul of each dilution was added to wells of a 96 well plate in triplicate. To each of these dilutions 0.5 ul of SARS-CoV-2 pseudotyped lentivirus (diluted in OptiMEM to 10 ul, MOI 1, titer estimated using infection of HEK293-ACE2 cells) was added and incubated for 30 minutes at room temperature. 3 wells contained 20 ul of OptiMEM and 3 wells contain 19.5 ul OptiMEM+0.5 ul pseudovirus serve as controls to normalize and calculate IC50. After 30 minutes 5000 HEK-ACE2 cells in 100 ul of DMEM+10% FBS were added to each well of the plate and incubated at 37° C. 5% CO₂ for 72 hours. After 72 hours the media was removed carefully without disrupting the cells and cells were trypsinized and analyzed by flow cytometry (Attune NxT, ThermoFisher) and EGFP fluorescence was recorded for each well.

The MFI for EGFP fluorescence of the triplicate wells was averaged and plotted against concentration of ACE2/mAb and a four-parameter non-linear regression was used to estimate IC50 of neutralization.

For Mucus Trapping, multiple particle tracking analysis of fluorescent SARS-CoV-2 VLPs in human airways mucus (AM) was performed. Briefly, solutions of fluorescent VLPs and ACE2-Fc or ACE2-(G₄S)₆-Fc were added to ˜10 μL of fresh, undiluted airway mucus in custom-made glass chambers. The samples were then incubated at 37° C. for ˜30 mins before microscopy. PBS and antibody CR3022 (final concentration 10 mg/ml) were used as negative and positive controls, respectively. The same AM was used for all runs to allow direct comparison among samples. Videos of VLPs diffusing in AM were recorded with MetaMorph software (Molecular Devices, Sunnyvale, Calif.) at a temporal resolution of 66.7 ms. Videos were analyzed using NetTracker from AI Tracking Solutions to convert video raw data to particle traces. Time-averaged mean-squared displacements (MSDs) and effective diffusivity were calculated by transforming particle centroid coordinates were transformed into time MSDs with the formula <Δr²(τ)>=[x(t+τ)−x(t)]²+[y(t+τ)−y(t)]², where τ=time scale or time lag.

The hamster study referred to herein for evaluation of ACE2-(G₄S)₆-Fc was performed in golden Syrian hamster model of SARS-CoV-2 infection as described previously with some modifications. Four groups (n=8 per group) were dosed intra-nasally with ACE2-(G₄S)₆-Fc either as a prophylactic (4 hours before exposure) or as therapeutic (4, 24, or 48 hours post-challenge). One group dosed with PBS served as negative control. Viral challenge was performed by inoculating the mice with 100 uL of SARS-CoV-2 diluted in Dubelcco's modified Eagle medium intra-nasally. Hamsters were then dosed daily with ACE2-(G₄S)₆-Fc until they were sacrificed, 4 days after viral exposure. Viral load was quantified in nasal turbinate by qRT-PCR and normalized by β-actin (internal gene control).

For the nebulization study. ACE2-(G₄S)₆-Fc in standard buffer at 10 mg/mL, was nebulized using a Phillips Innospire Go vibrating mesh nebulizer. Aerosols were collected into a glass impinger setup with upper and lower chambers, following protocol guidance in European Pharmacopoeia 5.0. The nebulizer was run until it was visually dry. Then, buffer was added to the different chambers of the glass impinger to recover the deposited antibodies. Aggregate formation in the upper chamber, lower chamber, and left-over (“dead volume”) samples was assessed by SEC using a EN-Rich 650 size exclusion column (Bio-Rad) mounted on a NGC Quest 10 FPLC (BioRad) and native PAGE as described previously. Binding affinity of nebulized molecules were assessed by S-protein ELISA as described above.

### Results

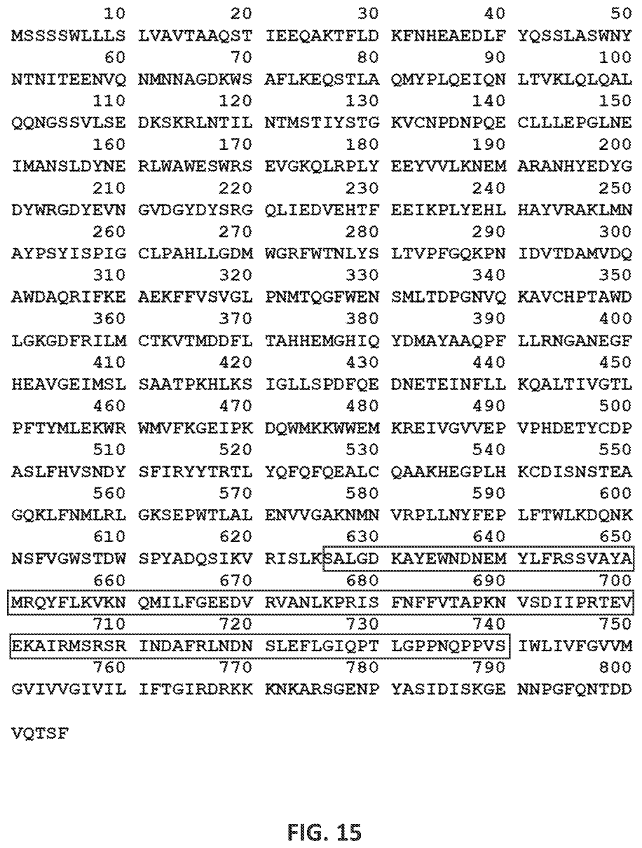

The stability of the molecules using differential scanning calorimetry was examined. The melting temperature T_{M} for ACE2-(G₄S)₆-Fc is ˜52±0.6° C. (See, e.g., FIG. 14).

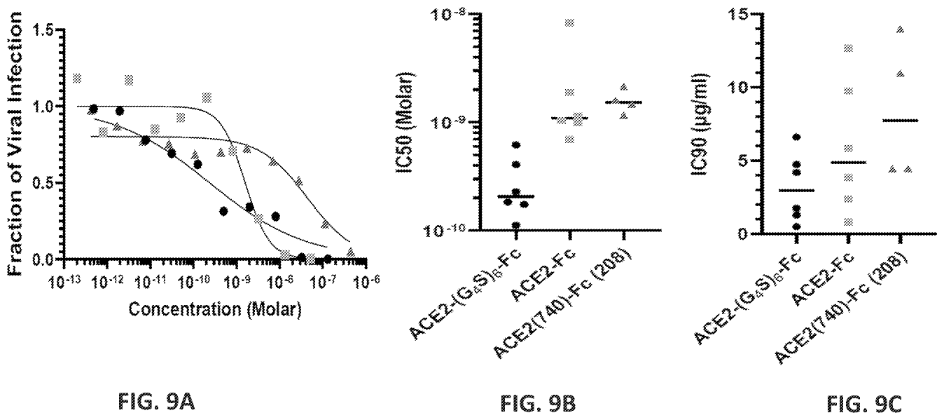

The potencies of different ACE2 decoys were determined by first measuring the binding affinity of the different ACE2 decoys to the spike protein of WT strain USA-WA1/2020 using ELISA. In addition to ACE2-Fc and ACE2-(G₄S)₆-Fc, full length ACE2-decoy (i.e. ACE2(740)-Fc, abbreviated as 208) were examined. Among the three ACE2-decoys, ACE2-(G₄S)₆-Fc consistently displayed the highest binding affinity, as shown in FIG. 8A. Across multiple independently produced batches, ACE2-(G₄S)₆-Fc consistently exhibited picomolar EC₅₀ (mean: 490 pM, or 96 ng/mL, see e.g., FIG. 8B); the median EC₅₀ with the most potent batch of ACE2-(G₄S)₆-Fc was as low as 136 pM, or 27 In contrast, the mean EC₅₀ with ACE2-Fc and ACE2(740)-Fc, at 3.6 nM (680 ng/mL) and 1.6 nM (370 ng/mL), was ˜7.3-fold and ˜3.3-fold worse than ACE2-(G₄S)₆-Fc.

ACE2-(G₄S)₆-Fc was produced in Chinese Hamster Ovary (CHO) cells, the most commonly utilized cells for large scale biologics production, with comparable EC₅₀ (see, e.g., FIG. 8C) as ACE2-(G₄S)₆-Fc produced in Expi293 cells.

In general, the flexibly linked ACE2 decoys described herein may be more likely than conventional monoclonal antibodies (mAbs) to bind different SARS-CoV-2 variants. Binding affinity experiments confirmed that ACE2-(G₄S)₆-Fc can indeed bind different SARS-CoV-2 variants using B.1.1.7 (UK) and B.1.351 (SA) spike proteins using ELISA, as shown in FIG. 8C. Across 5 independently produced ACE2-(G₄S)₆-Fc batches, their binding affinity to WT and U.K. and S.A. variants were highly comparable (FIG. 8D). For comparison, the binding of RGN10989, a mAb developed by Regeneron that is part of the mAb cocktail that received EUA from the FDA was also examined. While RGN10989 was able to bind WT and UK S proteins with comparable binding affinity, the mAb failed to achieve detectible binding against the SA S protein. These results underscore the utility of the flexibly linked ACE2 decoys described herein (including, but not limited to ACE2-(G₄S)₆-Fc) across all viruses that bind to ACE2, such as SARS-CoV-2 variants.

The increased apparent binding affinity of ACE2-(G₄S)₆-Fc also correlates with greater neutralizing activity. Neutralization potencies of ACE2-(G₄S)₆-Fc, ACE2-Fc and ACE2(740)-Fc were measured via standard pseudovirus assay, where HEK cells overexpressing ACE2 is infected with lentivirus encoding eGFP transgene pseudotyped with D614G variant of SARS-CoV-2 spike protein. The infectivity of the pseudovirus at different ACE2-decoy concentrations can be determined by using flow cytometry to measure eGFP fluorescence of cells incubated with varying amounts of ACE2 decoys. In this assay setup, ACE2-(G₄S)₆-Fc neutralized the SARS-CoV-2 pseudovirus with picomolar affinity, with an average IC₅₀ of 52 ng/mL. In contrast, the neutralization potency of ACE2-Fc and ACE2(740)-Fc was nearly 5-fold and 6-fold reduced, with IC₅₀ ˜240 ng/mL, and ˜310 ng/mL, respectively, ACE2-(G₄S)₆-Fc also possessed ˜2-fold greater IC₉₀ than ACE2-Fc (˜2.3 μg/ml vs ˜4.2 μg/ml). These results confirmed ACE2-(G₄S)₆-Fc indeed possess greater binding and affinity and neutralization potency than conventional ACE2-decoys.

The flexibly linked ACE2 decoys described herein, such as ACE2-(G₄S)₆-Fc, effectively trap viruses that bind to ACE2, such as SARS-CoV-2, VLPs in human airway mucus and can be stably nebulized. SARS-CoV-2, just like SARS-CoV-1, N1_63 and HKU1 coronaviruses, infects strictly via the apical side of airway epithelium (i.e. airway lumen), and predominantly shed progeny viruses back into airway mucus (AM), as the infection spread from the upper respiratory tract to the lower respiratory tract, with no appreciable shedding basally or cell-to-cell spread. This mechanism of viral spread implies that viruses must diffuse across AM for the infection to propagate within the airways. In turn, preventing viruses from diffusing across AM by crosslinking the viruses to the mucin matrix of AM may help arrest the spread of infection, and facilitates rapid clearance from the airways via natural mucociliary clearance mechanisms. This may allow potent trapping of viruses in human AM, leading to rapid clearance of VLPs from the airways.

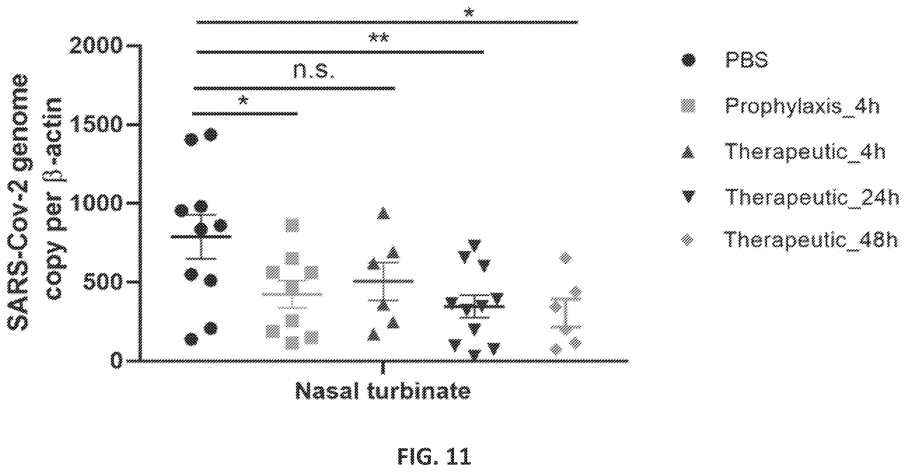

To evaluate whether the flexibly linked ACE2 decoys described herein (such as, but not limited to ACE2-(G₄S)₆-Fc) can trap SARS-CoV-2 in human AM, fluorescent SARS-2 VLP was prepared by co-expressing S protein with GAG-mCherry fusion construct, and its mobility in fresh human AM isolated from extubated endotracheal tubes was visualized. As shown in FIGS. 10A and 10B, ACE2-(G₄S)₆-Fc effectively trapped SARS-2 VLP in AM, reducing the fast moving viral populations (defined as possessing sufficient diffusivity to diffuse across ˜50 um layer in ˜1 hr) by ˜14 fold vs. saline control even at just 1 ug/mL cone in AM. In contrast, neither ACE2-Fc or CR3022, a high affinity mAb against S protein from JNJ/Crucell, were able to reduce viral mobility to the same extent even at 10×higher concentrations.

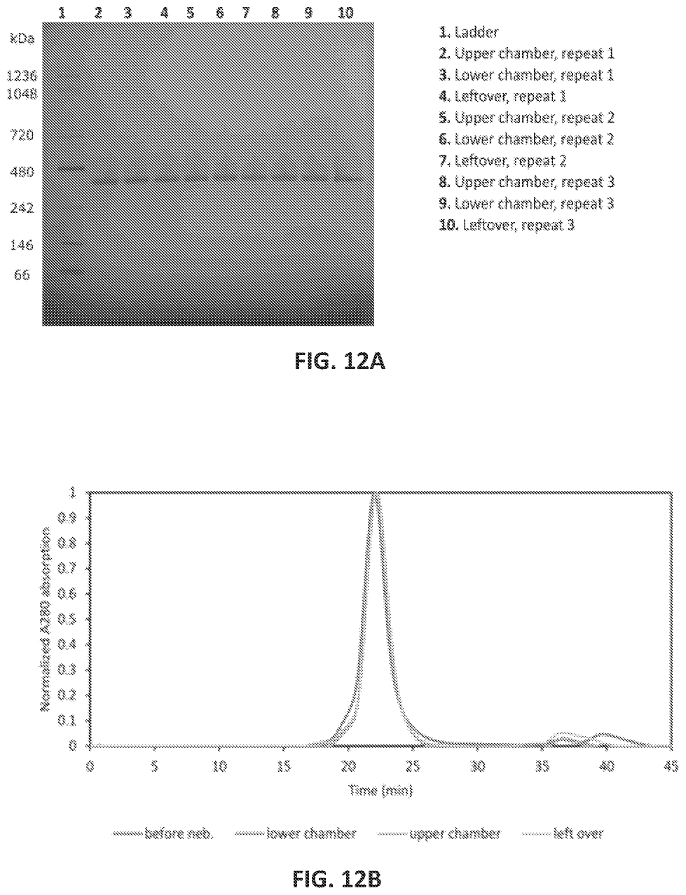

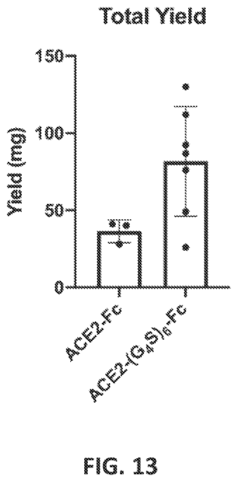

The most direct method to achieve therapeutic concentrations of mAb in the respiratory tract, particularly the lung airways, is to directly deliver the mAb via inhalation. Vibrating mesh nebulizers are capable of nebulizing protein therapeutics without generating local heating and shearing that can degrade proteins. The flexibly linked ACE2 decoys described herein can be stably nebulized. For example, a Philip's Innospire Go vibrating mesh nebulizer was used to nebulized ACE2-(G₄S)₆-Fc, collected the resulting aerosols in a two-chamber glass impinger setup designed to capture aerosols >6 μm (upper chamber) and <6 μm (lower chamber) following European Pharmacopoeia 5.0, and measured the binding affinity of the recovered nebulized ACE2-(G₄S)₆-Fc via S-protein ELISA. The results are shown in FIG. 11. No appreciable loss in binding affinity of ACE2-(G₄S)₆-Fc recovered from either the upper or lower chamber was observed, compared to ACE2-(G₄S)₆-Fc that was not nebulized. Native PAGE also confirmed there is no separation of the heavy chains or detectible aggregation (see, e.g., FIGS. 12A-12B). These results underscore the ability to stably nebulize flexibly linked. ACE2 decoys for direct inhalation delivery into the respiratory tract.

In addition, intranasal delivery of flexibly linked ACE2 decoys reduces viral load in the nasal turbinates. For example, hamsters infected with SARS-CoV-2 showed a reduction in viral load after treatment with flexibly linked ACE2 decoys. As an in vivo proof-of-concept, the efficacy of intrasal delivery of ACE2-(G₄S)₆-Fc in Golden Syrian Hamsters infected with live SARS-CoV-2 was assayed. Hamsters presents clinical signs of weight loss, and histopathological changes with high viral loads in the lungs, making them a suitable model for testing mAb-based approaches despite differences in anatomy of the respiratory tract. Most prior studies evaluated mAb against SARS-CoV-2 dosed within 2-6 hrs following infection. Here, initating daily dosing ACE2-(G₄S)₆-Fc was evaluated either before infection or at 4 hrs, 24 hrs and 48 hrs post-infection. ACE2-(G₄S)₆-Fc treatment, even when delayed until 48 hrs post-infection, provided a ˜10-fold reduction in viral load in the nasal turbinate tissues by 96 hrs. This translated to substantial reduction in weight loss over just the 2 day period (p=0.03).

Despite the remarkable potencies of many of mAbs advanced into clinical studies, viral escape mutants can readily develop against any individual mAb, with escape mutants still retaining ACE2 binding. To prevent escape mutants, many groups have focused on combining two antibodies targeting distinct structural epitopes. Although the risks of viral escape can be greatly reduced through the use of such mAb cocktails, it remains possible for viral escape mutants to simultaneously escape from both mAbs in a cocktail. In light of the concerns on viral escape mutants, and the enormous costs and time needed to advance a mAb molecule through Phase 3 clinical studies, it is exceedingly advantageous to develop a binding protein that is not at risk of viral escape. Furthermore, given that there are already at least 3 human coronaviruses that target ACE2 as the primary host entry receptor, including two with pandemic potential (SARS-CoV-1, SARS-CoV-2), it is likely simply a matter of time before another respiratory virus that also targets ACE2 emerges with pandemic potential. For these reasons, the flexibly-linked ACE2 decoys described herein may enable am immunotherapy against all ACE2-targeted viruses. Indeed, the soluble flexibly linked ACE2 decoys described herein can block infections by both SARS-CoV-1 and SARS-CoV2, and are able to bind S proteins from WT, UK and SA strains of the SARS-CoV-2 with comparable affinities (see, e.g., FIGS. 8C and 8D).

Although the flexibly linked ACE2 decoys described herein may use a wildtype (WI) ACE2 fragment, which may reduce the potential risk of escape viral mutants that bind WT ACE2 but cannot be captured by the ACE2 mutant, in some cases mutated ACE2 may be used, as described herein. In addition, the Fc domain may be wildtype (e.g., IgG1-Fc) or modified. In general, the collectrin domain may be omitted, and the linkage between the extracellular fragment of ACE2 with the Fc IgG1-Fc) domain may be optimized so that the length of the linker region allows multi-valent binding. These binding molecules have substantially better binding affinity and neutralization potencies compared to either the full length ACE2 with the collectrin domain, or ACE2-Fc conjugates without the flexible linker, with picomolar binding affinity and inhibitory concentrations (IC₅₀ ˜52 ng/mL) that rival or surpass ACE2-decoys that lack the flexible linker of sufficient length as described herein.

ACE2 dimerizes via its collectrin domain on the cell surface. The flexibly-linked ACE2 decoys described herein specifically removed the collectrin domain of ACE2, which enable the extracellular fragment of ACE2 to be grafted to wildtype Fc with well-defined linkers as described herein. In examples of the flexibly-linked ACE2 decoys described herein in which wildtype Fc was used (such as ACE2-(G₄S)₆-Fc) the constructs may also facilitate other cell-mediated immunity. Surprisingly, the flexibly-linked ACE2 decoys described herein also resulted in greater yield and stability; for example, the yield of ACE2-(G₄S)₆-Fc is comparable to other highly expressing IgGs produced under similar conditions, with a reproducible monomeric profile (in contrast to other ACE2-decoys that readily aggregate).

ACE2 can only bind to S proteins that have their RBD in the “up” conformation. Consequently, two RBD domains would be required to be in “up” conformation to achieve bivalent binding. S proteins with RBD domains in the “2-up” conformation have not been observed during imaging of SARS-CoV-2 S protein; however, it has been suggested that binding to an RBD can trigger transitioning of the S protein into a “3-up” state, a mechanism conserved among Coronaviridae. Mutations in different regions of the S protein can increase the proportion of S proteins with RBD domains in the “2-up” or “3-up” conformation. For instance, S protein with D614G showed a higher proportion of molecules in “2-up” and “3-up” conformation than. D614. Hexa-pro mutations also increase the number of S proteins with two RBDs in the up orientation. Other mutations that increased the ratio of “2-up” to “1-up” S proteins have been reported. Consequently, intra-spike binding to SARS-CoV-2 can be achieved, and we expect higher neutralization of SARS-CoV-2 with mutations that increase RBD exposure such us D614G.

There are important advantages to topical inhaled delivery. First, inhaled delivery maximizes the local concentrations in the lung and minimizes total dose of agent (e.g., mAb) needed compared to systemic dosing, as usually only a very small fraction of the systemically dosed Ab actually distributes into the airways. For the same amount of agent (e.g., binding agents such as mAbs), topical delivery can likely treat 4-10× more patients compared to systemic delivery while still achieving greater concentrations in the lung. This both reduces the cost burden, and more importantly allows us to potentially treat many more patients, an essential consideration given the nearly unprecedented scale of COVID-19. Second, early treatment is highly desirable, a universal fact for all antivirals. Unfortunately, for systemically dosed agent or small molecule drugs, even when given quickly following diagnosis, there is significant delay before the agent can reach Cmax in the lung. For instance, it takes 3 days of twice-daily oseltamivir dosing to achieve steady-state concentrations in the lung. In contrast, nebulization delivers the flexibly-linked ACE2 decoys described herein ACE2-(G₄S)₆-Fc directly into the airways, thus enable local Cmax to be reached quickly. Nebulization also bypasses the need for infusion chairs and post-infusion monitoring, and enables therapy to take place directly in the comfort of the patients' own home. This greatly reduce the burden on the healthcare infrastructure to administer the treatment compared to IV delivery, which generally requires ˜1-2 hrs of infusion followed by a comparable duration of post-infusion observation.

AM is continuously secreted into the lung airways each day, which are transported from the lower airways (bronchioles) to the trachea by natural mucociliary or cough driven clearance, before being swallowed subconsciously at the esophagus for sterilization by the acidic and degradative gastric environment. Natural mucus clearance quickly removes any foreign particulates that are deposited along the lung airways. Respiratory viruses must diffuse through AM to spread, and have specifically evolved to do so efficiently. By crosslinking viruses to mucins using binding proteins, we not only ensure the viruses cannot diffuse through mucus to spread the infection, but also directly remove the virus and associated antigens from the airways. This in turn minimizes the potential inflammation and antigen-directed immune response that can occur by macrophages and neutrophils that can infiltrate into the lung. The flexibly-linked. ACE2 decoys described herein may be used with only a single dose, e.g., once per day.

SEQUENCE LISTING

SEQ ID NO: 1
(Signal Peptide)
MSSSSWLLLSLVAVTAA
SEQ ID NO: 2
(ACE2 with H374N + H378N)
QSTIEEQAKTFLDKFNHEAEDLFYQSSLASWNYNTNITEENVQNMNNAGDKWSAFLKEQSTLAQ
MYPLQEIQNLTVKLQLQALQQNGSSVLSEDKSKRLNTILNTMSTIYSTGKVCNPDNPQECLLLE
PGLNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMWGR
FWTNLYSLTVPFGQKPNIDVTDAMVDQAWDAQRIFKEAEKFFVSVGLPNMTQGFWENSMLTDPG
NVQKAVCHPTAWDLGKGDFRILMCTKVTMDDFLTAHNEMGNIQYDMAYAAQPFLLRNGANEGFH
EAVGEIMSLSAATPKHLKSIGLLSPDFQEDNETEINFLLKQALTTVGTLPFTYMLEKWRWMVFK
GEIPKDQWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDQNKNSFVGWSTDWSPYAD
SEQ ID NO: 3
(G4S6 + Hinge + Fc)
GGGGSGGGGSGGGGSGGGGSGGGGSGGGGSEPKSCDKTHTCPPCPAPELLGGPSVFLFPPKPKD
TLMISRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEALHNHYTQKSL
SLSPGK*
SEQ ID NO: 4
(Hinge + Fc)
EPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSDGSFFLY
SKLTVDKSRWQQGNVESCSVMHEALHNHYTQKSLSLSPGK*
SEQ ID NO: 5
(Signal Peptide)
MKWVTFISLLFLFSSAYSGS
SEQ ID NO: 6
(CR3022 Light Chain)
DIQLTQSPDSLAVSLGERATINCKSSQSVLYSSINKNYLAWYQQKPGQPPKLLIYWASTRESGV
PDRFSGSGSGTDFTLTISSLQAEDVAVYYCQQYYSTPYTFGQGTKVEIKRTVAAPSVFIFPPSD
EQLKSGTASVVCLLNNFYPREAKVQWKVDNALQSGNSQESVTEQDSKDSTYSLSSTLTLSKADY
EKHKVYACEVTHQGLSSPVTKSFNRGEC*
SEQ ID NO: 7
(ACE2 + Linker)
QSTIEEQAKTFLDKFNHEAEDLFYQSSLASWNYNTNITEENVQNMNNAGDKWSAFLKEQSTLAQ
MYPLQEIQNLTVKLQLQALQQNGSSVLSEDKSKRLNTILNTMSTIYSTGKVCNPDNPQECLLLE
PGLNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMWGR
FWTNLYSLTVPFGQKPNIDVTDAMVDQAWDAQRIFKEAEKFFVSVGLPNMTQGFWENSMLTDPG
NVQKAVCHPTAWDLGKGDFRILMCTKVTMDDFLTAHHEMGHIQYDMAYAAQPFLLRNGANEGFH
EAVGEIMSLSAATPKHLKSIGLLSPDFQEDNETEINFLLKQALTIVGTLPFTYMLEKWRWMVFK
GEIPKDQWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDQNKNSFVGWSTDWSPYADGGSGGGGSGGGGSGGGGSGGGGSGGGGSGGGGS
SEQ ID NO: 8
(CR3022VH + CH1 + Fc)
QMQLVQSGTEVKKPGESLKISCKGSGYGFITYWIGWVRQMPGKGLEWMGIIYPGDSETRYSPSE
QGQVTISADKSINTAYLQWSSLEASDTAIYYCAGGSGISTPMDVWGQGTTVTVSSASTKGPSVF
PLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLSLSPG*
SEQ ID NO: 9
(CR3022VH + CH1 + Fc)
QMQLVQSGTEVKKPGESLKISCKGSGYGFTTYWIGWVRQMPGKGLEWMGTTYPGDSETRYSPSF
QGQVTISADKSINTAYLQWSSLKASDTAIYYCAGGSGISTPMDVWGQGTTVTVSSASTKGPSVF
PLAPSSKSTSGGTAALGCLVKDYFPEPVTVSWNSGALTSGVHTFPAVLQSSGLYSLSSVVTVPS
SSLGTQTYlCNVNHKPSNTKVDKRVEPKSCDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLSLSP
SEQ ID NO: 10
(Linker + ACE2)
GGSGGGGSGGGGSGGGGSGGGGSGGGGSGGGGSEFQSTIEEQAKTFLDKFNHEAEDLFYQSSLA
SWNYNTNITEENVQNMNNAGDKWSAFLKEQSTLAQMYPLQEIQNLTVKLQLQALQQNGSSVLSE
DKSKRLNTILNTMSTIYSTGKVCNPDNPQECLLLEPGLNEIMANSLDYNERLWAWESWRSEVGK
QLRPLYEEYVVLKNEMARANHYEDYGDYWRGDYEVNGVDGYDYSRGQLIEDVEHTFEEIKPLYE
HLHAYVRAKLMNAYPSYISPIGCLPAHLLGDMWGRFWTNLYSLTVPFGQKPNIDVTDAMVDQAW
DAQRIFKEAEKFFVSVGLPNMTQGFWENSMLTDPGNVQKAVCHPTAWDLGKGDFRILMCTKVTM
DDFLTAHHEMGHIQYDMAYAAQPFLLRNGANEGFHEAVGEIMSLSAATPKHLKSIGLLSPDFQE
DNETEINFLLKQALTIVGTLPFTYMLEKWRWMVFKGEIPKDQWMKKWWEMKREIVGVVEPVPHD
ETYCDPASLFHVSNDYSFIRYYTRTLYQFQFQEALCQAAKHEGPLHKCDISNSTEAGQKLFNML
RLGKSEPWTLALENVVGAKNMNVRPLLNYFEPLFTWLKDQNKNSFVGWSTDWSPYAD*
SEQ ID NO: 11
(ACE2 WT, aa 19-615, excludes collectrin domain)
STIEEQAKTFLDKFNHEAEDLFYQSSLASWNYNTNITEENVQNMNNAGDKWSAFLKEQSTLAQM
YPLQEIQNLTVKLQLQALQQNGSSVLSEDKSKRLNTILNTMSTIYSTGKVCNPDNPQECLLLEP
GLNEIMANSLDYNERLWAWESWRSEVGKQLRPLYEEYVVLKNEMARANHYEDYGDYWRGDYEVN
GVDGYDYSRGQLIEDVEHTFEEIKPLYEHLHAYVRAKLMNAYPSYISPIGCLPAHLLGDMWGRE
WTNLYSLTVPFGQKPNIDVTDAMVDQAWDAQRIFKEAEKFFVSVGLPNMTQGFWENSMLTDPGN
VQKAVCHPTAWDLGKGDFRILMCTKVTMDDFLTAHHEMGHIQYDMAYAAQPFLLRNGANEGFHE
AVGEIMSLSAATPKHLKSIGLLSPDFQEDNETEINFLLKQALTIVGTLPFTYMLEKWRWMVFKG
EIPKDQWMKKWWEMKREIVGVVEPVPHDETYCDPASLFHVSNDYSFIRYYTRTLYQFQFQEALC
QAAKHEGPLHKCDISNSTEAGQKLFNMLRLGKSEPWTLALENVVGAKNMNVRPLLNYFEPLFTW
LKDQNKNSFVGWSTDWSPYAD
SEQ ID NO: 12
(Fc heavy chain 1)
PCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQ
GNVFSCSVMHEALHNHYTQKSLSLSPG
SEQ ID NO: 13
(Fc heavy chain 2)
PCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDKSRWQQ
GNVFSCSVMHEALHNHYTQKSLSLSPG
SEQ ID NO: 14
(Hinge)
EPKSCDKTHTCP
SEQ ID NO: 15
313-(G4S)6-Fc
(ACE2 excluding collectrin domain and modifying residues K31F, N33D, H34S,
E35Q, and H345L)
QSTIEEQAKTFLDFFDSQAEDLFYQSSLASWNYNTNITEENVQNMNNAGDKWSAFLKEQSTLAQ
MYPLQEIQNLTVKLQLQALQQNGSSVLSEDKSKRLNTILNTMSTIYSTGKVCNPDNPQECLLLE
PGLNEIMANSLDYNERLWAWESWRSEVGKQLRPLYEEYVVLKNEMARANHYEDYGDYWRGDYEV
NGVDGYDYSRGQLLEDVEHTHEELKPLYEHLHAYVRAKLMNAYPSYISPLGCLPAHLLGDMWGR
FWTNLYSLTVPFGQKPNIDVTDAMVDQAWDAQRIFKEAEKFFVSVGLPNMTQGFWENSMLTDPG
NVQKAVCLPTAWDLGKGDFRILMCTKVTMDDFLTAHHEMGHIQYDMAYAAQPFLLRNGANEGFH
EAVGEIMSLSAATPKHLKSIGLLSPDFQEDNETEINFLLKQALTTVGTLPFTYMLEKWRWMVFK
GEIPKDQWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDQNKNSFVGWSTDWSPYAD
SEQ ID NO: 16
313-(G4S)6-Fc
(ACE2 excluding collectrin domain and modifying residues K31F, N33D, H34S,
E35Q, and H345L + (G4S)6 linker + Hinge + Fc). May optionally include a GS
in front of linker.
QSTIEEQAKTFLDFFDSQAEDLFYQSSLASWNYNTNITEENVQNMNNAGDKWSAFLKEQSTLAQ
MYPLQEIQNLTVKLQLQALQQNGSSVLSEDKSKRLNTILNTMSTIYSTGKVCNPDNPQECLLLE
PGLNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMWGR
FWTNLYSLTVPFGQKPNIDVTDAMVDQAWDAQRIFKEAEKFFVSVGLPNMTQGFWENSMLTDPG
NVQKAVCLPTAWDLGKGDFRILMCTKVTMDDFLTAHHEMGHIQYDMAYAAQPFLLRNGANEGFH
EAVGEIMSLSAATPKHLKSIGLLSPDFQEDNETEINFLLKQALTIVGTLPFTYMLEKWRWMVFK
GEIPKDQWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDQNKNSFVGWSTDWSPYADGGGGSGGGGSGGGGSGGGGSGGGGSGGGGSEPKSCDKTHTCP
PCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDKSRWQQ
GNVFSCSVMHEALHNHYTQKSLSLSPG
SEQ ID NO: 17
sACE2.v2.4-8h-(G4S)6-Fc
(ACE2 excluding collectrin domain and modifying residues T27Y, L79T, N330Y)
QSTIEEQAKYFLDKFNHEAEDLFYQSSLASWNYNTNITEENVQNMNNAGDKWSAFLKEQSTTAQ
MYPLQEIQNLTVKLQLQALQQNGSSVLSEDKSKRLNTILNTMSTIYSTGKVCNPDNPQECLLLE
PGLNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMWGR
FWTNLYSLTVPFGQKPNIDVTDAMVDQAWDAQRIFKEAEKFFVSVGLPNMTQGFWEYSMLTDPG
NVQKAVCHPTAWDLGKGDFRILMCTKVTMDDFLTAHHEMGHIQYDMAYAAQPFLLRNGANEGFH
EAVGEIMSLSAATPKHLKSIGLLSPDFQEDNETEINFLLKQALTIVGTLPFTYMLEKWRWMVFK
GEIPKDQWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDQNKNSFVGWSTDWSPYAD
SEQ ID NO: 18
sACE2.v2.4-8h-(G4S)6-Fc
(ACE2 excluding collectrin domain and modifying residues T27Y, L79T, N330Y +
(G4S)6 linker + hinge + Fc). May optionally include a GS in front of linker.
QSTIEEQAKYFLDKFNHEAEDLFYQSSLASWNYNTNITEENVQNMNNAGDKWSAFLKEQSTTAQ
MYPLQEIQNLTVKLQLQALQQNGSSVLSEDKSKRLNTILNTMSTIYSTGKVCNPDNPQECLLLE
PGLNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMWGR
FWTNLYSLTVPFGQKPNIDVTDAMVDQAWDAQRIFKEAEKFFVSVGLPNMTQGFWEYSMLTDPG
NVQKAVCHPTAWDLGKGDFRILMCTKVTMDDFLTAHHEMGHIQYDMAYAAQPFLLRNGANEGFH
EAVGEIMSLSAATPKHLKSIGLLSPDFQEDNETEINFLLKQALTIVGTLPFTYMLEKWRWMVFK
GEIPKDQWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDQNKNSFVGWSTDWSPYADGGGGSGGGGSGGGGSGGGGSGGGGSGGGGSEPKSCDKTHTCP
PCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTTSKAKGQPREPQVYTLPPSR
EEMTKNQVSLTCLVKGFYPSDIAVEWESNGQPENNYKTTPPVLDSDGSFFLYSKLTVDKSRWQQ
GNVFSCSVMHEALHNHYTQKSLSLSPG
SEQ ID NO: 19
3J320v2-(G4S)6-Fc
(ACE2 excluding collectrin domain and modifying residues T20I, H34A, T92Q,
and Q101H)
QSIIEEQAKTFLDKFNAEAEDLFYQSSLASWNYNTNITEENVQNMNNAGDKWSAFLKEQSTLAQ
MYPLQEIQNLQVKLQLQALHQNGSSVLSEDKSKRLNTILNTMSTIYSTGKVCNPDNPQECLLLE
PGLNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMWGR
FWTNLYSLTVPFGQKPNIDVTDAMVDQAWDAQRIFKEAEKFFVSVGLPNMTQGFWENSMLTDPG
NVQKAVCHPTAWDLGKGDFRILMCTKVTMDDFLTAHHEMGHIQYDMAYAAQPFLLRNGANEGFH
EAVGEIMSLSAATPKHLKSIGLLSPDFQEDNETEINFLLKQALTIVGTLPFTYMLEKWRWMVFK
GEIPKDQWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDQNKNSFVGWSTDWSPYA
SEQ ID NO: 20
3J320v2-(G4S)6-Fc
(ACE2 excluding collectrin domain and modifying residues T20I, H34A, T92Q, and
Q101H + (G4S)6 linker + hinge + Fc). May optionally include a GS in front of
linker.
QSIIEEQAKTFLDKFNAEAEDLFYQSSLASWNYNTNITEENVQNMNNAGDKWSAFLKEQSTLAQ
MYPLQEIQNLQVKLQLQALRQNGSSVLSEDKSKRLNTILNTMSTIYSTGKVCNPDNPQECLLLE
PGLNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMWGR
FWTNLYSLTVPFGQKPNIDVTDAMVDQAWDAQRIFKEAEKFFVSVGLPNMTQGFWENSMLTDPG
NVQKAVCHPTAWDLGKGDFRILMCTKVTMDDFLTAHHEMGHIQYDMAYAAQPFLLRNGANEGFH
EAVGEIMSLSAATPKHLKSIGLLSPDFQEDNETEINFLLKQALTIVGTLPFTYMLEKWRWMVFK
GEIPKDQWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDQNKNSFVGWSTDWSPYAGGGGSGGGGSGGGGSGGGGSGGGGSGGGGSEPKSCDKTHTCPP
CPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLTVDKSRWQQG
NVFSCSVMHEALHNHYTQKSLSLSPG
SEQ ID NO: 21
3N39v2-(G4S)6-Fc
(ACE2 excluding collectrin domain and modifying residues A25V, K31N, E34K and
L79F)
QSTIEEQVKTFLDNFNHKAEDLFYQSSLASWNYNTNITEENVQNMNNAGDKWSAFLKEQSTPAQ
MYPLQEIQNLTVKLQLQALQQNGSSVLSEDKSKRLNTILNTMSTIYSTGKVCNPDNPQECLLLE
PGLNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMWGR
FWTNLYSLTVPFGQKPNIDVTDAMVDQAWDAQRIFKEAEKFFVSVGLPNMTQGFWENSMLTDPG
NVQKAVCHPTAWDLGKGDFRILMCTKVTMDDFLTAHHEMGHIQYDMAYAAQPFLLRNGANEGFH
EAVGEIMSLSAATPKHLKSIGLLSPDFQEDNETEINFLLKQALTIVGTLPFTYMLEKWRWMVFK
GEIPKDQWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDQNKNSFVGWSTDWSPYA
SEQ ID NO: 22
3N39v2-(G4S)6-Fc
(ACE2 excluding collectrin domain and modifying residues A25V, K31N, E34K and
L79F + (G4S)6 linker + hinge + Fc). May optionally include a GS in front of
linker.
QSTIEEQVKTFLDNFNHKAEDLFYQSSLASWNYNTNITEENVQNMNNAGDKWSAFLKEQSTPAQ
MYPLQEIQNLTVKLQLQALQQNGSSVLSEDKSKRLNTILNTMSTIYSTGKVCNPDNPQECLLLE
PGLNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMWGR
FWTNLYSLTVPFGQKPNIDVTDAMVDQAWDAQRIFKEAEKFFVSVGLPNMTQGFWENSMLTDPG
NVQKAVCHPTAWDLGKGDFRILMCTKVTMDDFLTAHHEMGHIQYDMAYAAQPFLLRNGANEGFH
EAVGEIMSLSAATPKHLKSIGLLSPDFQEDNETEINFLLKQALTIVGTLPFTYMLEKWRWMVFK
GEIPKDQWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDQNKNSFVGWSTDWSPYAGGGGSGGGGSGGGGSGGGGSGGGGSGGGGSEPKSCDKTHTCPP
CPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLTVDKSRWQQG
NVFSCSVMHEALHNHYTQKSLSLSPG
SEQ ID NO: 23
ACE2615-foldon-T27W-(G₄S)₆-Fc
(ACE2 modifying residue T27W)
QSTIEEQAKWFLDKFNHEAEDLFYQSSLASWNYNTNITEENVQNMNNAGDKWSAFLKEQSTLAQ
MYPLQEIQNLTVKLQLQALQQNGSSVLSEDKSKRLNTILNTMSTIYSTGKVCNPDNPQECLLLE
PGLNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMWGR
FWTNLYSLTVPFGQKPNIDVTDAMVDQAWDAQRIFKEAEKFFVSVGLPNMTQGFWENSMLTDPG
NVQKAVCHPTAWDLGKGDFRILMCTKVTMDDFLTAHHEMGHIQYDMAYAAQPFLLRNGANEGFH
EAVGEIMSLSAATPKHLKSIGLLSPDFQEDNETEINFLLKQALTIVGTLPFTYMLEKWRWMVFK
GEIPKDQWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDQNKNSFVGWSTDWSPYAD
SEQ ID NO: 24
ACE2615-foldon-T27W-(G₄S)₆-Fc
(ACE2 modifying residue T27W + (G4S)6 linker + hinge + Fc). May optionally
include a GS in front of linker.
QSTIEEQAKWFLDKFNHEAEDLFYQSSLASWNYNTNITEENVQNMNNAGDKWSAFLKEQSTLAQ
MYPLQEIQNLTVKLQLQALQQNGSSVLSEDKSKRLNTILNTMSTIYSTGKVCNPDNPQECLLLE
PGLNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMWGR
FWTNLYSLTVPFGQKPNIDVTDAMVDQAWDAQRIFKEAEKFFVSVGLPNMTQGFWENSMLTDPG
NVQKAVCHPTAWDLGKGDFRILMCTKVTMDDFLTAHHEMGHIQYDMAYAAQPFLLRNGANEGFH
EAVGEIMSLSAATPKHLKSIGLLSPDFQEDNETEINFLLKQALTIVGTLPFTYMLEKWRWMVFK
GEIPKDQWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDQNKNSFVGWSTDWSPYADGGGGSGGGGSGGGGSGGGGSGGGGSGGGGSEPKSCDKTHTCP
PCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDKSRWQQ
GNVFSCSVMHEALHNHYTQKSLSLSPG
SEQ ID NO: 25
LCB1
DKEWILQKIYEIMRLLDELGHAEASMRVSDLIYEFMKKGDERLLEEAERLLEEVER
SEQ ID NO: 26
LCB1-(G₄S)₆-Fc
May optionally include a GS in front of linker.
DKEWILQKIYEIMRLLDELGHAEASMRVSDLIYEFMKKGDERLLEEAERLLEEVERGGGGSGGG
GSGGGGSGGGGSGGGGSGGGGSEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKSLSLSPG
SEQ ID NO: 27
LCB3
May optionally include a GS in front of linker.
NDDELHMLMTDLVYEALHFAKDEEIKKRVFQLFELADKAYKNNDRQKLEKVVEELKELLERLLS
SEQ ID NO: 28
LCB3-(G4S)6-Fc
May optionally include a GS in front of linker.
NDDELHMLMTDLVYEALHFAKDEEIKKRVFQLFELADKAYKNNDRQKLEKVVEELKELLERLLS
GGGGSGGGGSGGGGSGGGGSGGGGSGGGGSEPKSCDKTHTCPPCPAPELLGGPSVFLFPPKPKD
TLMISRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEALHNHYTQKSL
SLSPG

What is claimed is:

1. An isolated binding protein that binds to ACE2-targeted viruses having an amino acid sequence comprising:
A-(B)ₙ-C (Formula I)
wherein
A is an extracellular portion of angiotensin-converting enzyme 2 (ACE2) excluding the collectrin domain, or a variant thereof;
n is 1, 2, 3, 4, 5, 6, 7, 8, 9, 10, 11, 12, 13, 14, 15, 16, 17, 18, 19, 20, 21, 22, 23, 24 or 25;
B is a polypeptide flexible linker;
C is a fragment crystallization (Fc) domain,
wherein the isolated binding protein dimerizes.

2. The binding protein of claim 1, wherein n is selected such that the distance between the A domains of the dimers is greater than 14 nm.

3. The binding protein of any of claims 1-2, wherein the Fc domain is a human IgA, IgM or IgG Fc domain.

4. The binding protein of claim 2, wherein the Fc domain is a human IgG1 Fc domain.

5. The binding protein of any of claims 1-3 wherein the Fc domain comprises a YTE mutation, an LS mutation, or a LALA-PG mutation.

6. The binding protein of any of claims 1-5, wherein the extracellular portion of ACE2 is an extracellular portion of a human ACE2.

7. The binding protein of any of claims 1-6, wherein the extracellular portion of ACE2 has an amino acid sequence identity of 80% or greater with the amino acid sequence of SEQ ID NO: 11.

8. The binding protein of any of claims 1-6, wherein the extracellular portion of ACE2 has an amino acid sequence that has up to 10 amino acid difference within the amino acid of SEQ ID NO: 11.

9. The binding protein of any of claims 1-6, wherein the extracellular portion of ACE2 comprises at least one mutation.

10. The binding protein of claim 9, wherein the ACE2 comprises two or more mutations.

11. The binding protein of any of claims 1-10, wherein the polypeptide flexible linker has the sequence of GGGGS.

12. The binding protein of any of claims 1-11, further comprising a hinge between the flexible linker and the Fc domain.

13. The binding protein of any of claims 1-12, wherein the Fc domain has an oligosaccharide having a G0 glycosylation pattern.

14. The binding protein of any of claim 1-13, wherein the Fc domain comprises an oligosaccharide having a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the binding protein in mucus.

15. A pharmaceutical composition comprising a binding protein of any of claims 1-14 and a pharmaceutically acceptable excipient.

16. The pharmaceutical composition of claim 15, wherein said excipient, or carrier is configured for inhalation.

17. The pharmaceutical composition of claim 15, wherein the composition is configured for one or more of: oral, parenteral, intraperitoneal, transmucosal, transdermal, rectal, inhalable, and topical administration.

18. A method of treating a subject suffering from SARS-CoV-2, the method comprising administering a pharmaceutically acceptable amount of the pharmaceutical composition of any of claims 15-17.

19. The method of claim 18, wherein administering comprises applying the pharmaceutical composition systemically to the patient.

20. The method of claim 18, wherein administering comprises applying the pharmaceutical composition to the patient's mucus membrane.

21. The method of claim 18, wherein administering comprises nebulizing the pharmaceutical composition.

22. A method of treating or inhibiting a viral infection by an ACE2-targeted virus, the method comprising administering to the subject, via an inhaled route, a binding protein of any of claims 1-14.

23. The method of claim 22 wherein the ACE2-targeted virus is SARS-CoV-2.

24. An isolated binding protein that binds to ACE2-targeted viruses having an amino acid sequence comprising:
A-(B)ₙ-C (Formula I)
wherein
A is an extracellular portion of angiotensin-converting enzyme 2 (ACE2) excluding the collectrin domain, having an amino acid sequence identity of 80% or greater with the amino acid sequence of SEQ ID NO: 11;
n is 1, 2, 3, 4, 5, 6, 7, 8, 9, 10, 11, 12, 13, 14, 15, 16, 17, 18, 19, 20, 21, 22, 23, 24 or 25;
B is a polypeptide flexible linker;
C is a fragment crystallization (Fc) domain,
wherein the isolated binding protein dimerizes,
further wherein n is selected such that the distance between the A domains of the dimers is greater than 14 nm.

25. A bispecific binding protein that hinds to severe acute respiratory syndrome coronavirus 2 (SARS-CoV-2) comprising at least one heavy chain variable region having a formula II:
X-(Y)ₙ-Z (Formula II)
wherein
X is (i) an angiotensin-converting enzyme 2 (ACE2) or a variant thereof; or (ii) a variable heavy chain region from an antibody that binds to SARS-CoV-2 or a fragment thereof;
n is 1, 2, 3, 4, 5, 6, 7, 8, 9, 10, 11, 12, 13, 14, 15, 16, 17, 18, 19, 20, 21, 22, 23, 24 or 25;
Y is a first polypeptide flexible linker;
and
Z is (i) ACE2 or a variant thereof; or (ii) a variable heavy chain region from an antibody that binds to SARS-CoV-2 or a fragment thereof, provided that when (a) X is ACE2 or a variant thereof, Z is a variable heavy chain region from an antibody that binds to SARS-CoV-2 or a fragment thereof; or (b) X is a variable heavy chain region from an antibody that hinds to SARS-CoV-2 or a fragment thereof, Z is ACE2 or a variant thereof.

26. The bispecific binding protein of claim 25 wherein when X or Z further comprise a fragment crystallization (Fc) domain, at least one heavy chain constant region from an antibody that hinds to SARS-CoV-2, at least one light chain constant region from an antibody that binds to SARS-CoV-2, at least one variable light chain region from an antibody that hinds to SARS-CoV-2 or any combinations thereof.

27. The bispecific binding protein of claim 26, wherein the Fc domain is a human IgA, IgM or IgG Fc domain.

28. The bispecific binding protein of claim 27 wherein the Fc domain is a human IgG1 Fc domain.

29. The bispecific binding protein of any of claims 25-28, wherein the ACE2 is a human ACE2.

30. The bispecific binding protein of any of claims 25-29, wherein the ACE2 comprises an extracellular domain of human ACE2.

31. The bispecific binding protein of any of claims 25-30, wherein the ACE2 comprises at least one mutation.

32. The bispecific binding protein of claim 31, wherein the ACE2 comprises two or more mutations.

33. The bispecific binding protein of any of claims 25-32, wherein the linker has the sequence of GGGGS.

34. The bispecific binding protein of any of claims 25-33, wherein the variable heavy chain region from an antibody is from monoclonal antibody CR3014 or CR3022.

35. The bispecific binding protein of any of claims 25-34, wherein the bispecific binding protein is a bispecific antibody or antibody binding fragment thereof.

36. A pharmaceutical composition comprising a bispecific binding protein of any of claims 25-35 and a pharmaceutically acceptable excipient.

37. A method of treating a subject suffering from SARS-CoV-2, the method comprising a pharmaceutically acceptable amount of the pharmaceutical composition of claim 36.
